SEQUENCE LISTING (5 sequences)

SEQ ID NO: 1 - protein, 16 aa, synthetic construct
  REGION 1..16: note = Synthetic
CYFDDSSNVLCKKYRS

SEQ ID NO: 2 - protein, 16 aa, synthetic construct
  REGION 1..16: note = Synthetic
CYYDNSSSVLCKRDRS

SEQ ID NO: 3 - protein, 16 aa, synthetic construct
  REGION 1..16: note = Synthetic
CYYDNSSSVLCKRYRS

SEQ ID NO: 4 - protein, 16 aa, synthetic construct
  REGION 1..16: note = Synthetic
  VARIANT 14: note = X can be R, H, K, D, E, S, T, N, Q, C, U, G, P, A, V, I, L, M, F, Y, W or D-Tyr
CYYDNSSSVLCKRXRS

SEQ ID NO: 5 - protein, 16 aa, synthetic construct
  REGION 1..16: note = Synthetic
CYYDNSSSVLCKKYRS

(19) United States
(12) Patent Application Publication
(10) Pub. No.: US 2024/0342216 A1
(43) Pub. Date: Oct. 17, 2024

(54) METHODS OF TREATING CANCER

(71) Applicant: Therapeutics By Design, LLC, Weston, MA (US)

(72) Inventors: William Dean Carlson, Weston, MA (US); Peter Keck, Weston, MA (US); Dattatreyamurty Bosukonda, Weston, MA (US); Frederic Roy Carlson, Jr., Weston, MA (US)

(21) Appl. No.: 18/613,828

(22) Filed: Mar. 22, 2024

Related U.S. Application Data
(63) Continuation of application No. PCT/US2022/044404, filed on Sep. 22, 2022.
(60) Provisional application No. 63/246,989, filed on Sep. 22, 2021.

(51) Int. Cl. A61K 35/17; A61K 38/18; A61K 39/00; A61K 45/06; A61P 35/00; C12N 5/0784

(52) U.S. Cl. CPC A61K 35/17; A61K 38/1875; A61K 39/4615; A61K 39/46443; A61K 45/06; A61P 35/00; C12N 5/0639; C12N 2501/22; C12N 2501/2304

(57) ABSTRACT

The present disclosure relates generally to methods of treating or preventing cancer, the method comprising administering to a subject in need of treatment at least one tissue differentiation factor related polypeptide (TDFRP), wherein the TDFRP is administered in an amount effective to treat the cancer in the subject.

### RELATED APPLICATIONS

This application is a Continuation of International Application Serial No. PCT/US2022/044404, filed on Sep. 22, 2022, which claims priority to U.S. Provisional Application No. 63/246,989, filed on Sep. 22, 2021. The entire contents of each of the foregoing applications are expressly incorporated by reference herein.

### SEQUENCE LISTING

The application contains a Sequence Listing which has been submitted electronically in .XML format and is hereby incorporated by reference in its entirety. Said .XML copy, created on Mar. 18, 2024, is named “132033-00420.xml” and is 7,524 bytes in size. The sequence listing contained in this .XML file is part of the specification and is hereby incorporated by reference herein in its entirety.

### BACKGROUND

Conventional treatment for cancers mainly targets bulk tumor cells; however, in a significant number of patients, cancer cells will acquire a drug resistant phenotype after standard therapies, resulting in metastasis and tumor recurrence after several years of treatment for which there is limited or no curative therapy. It has been suggested that, for the duration of this latency, disseminated tumor cells exist in distant secondary tissue sites as dormant micrometastases. Cancer stem cells (CSCs) are commonly identified as the culprits of metastatic relapse. This is due in part to the ability of CSCs to self-renew and generate malignant progeny that are usually resistant to chemotherapy and other therapeutic treatments.

Cancer stem cells represent a relatively small proportion of the cells within cancers and, through the classic stem cell processes of self-renewal and multi-lineage differentiation, can form new tumors containing all cell types found in the parent cancer tissue. They are defined experimentally by their ability to regrow tumors and are also referred to as tumorigenic cells. CSCs have a high propensity to initiate tumors upon transplantation and are the driving force behind malignancies. CSCs are shown to persist in tumors as a distinct population and cause relapse and metastasis. CSC rich tumors are associated with higher rates of metastasis and poor patient prognosis.

Growing evidence suggests deregulation of pathways that regulate self-renewal in cancer stem cells result in the continuous expansion of self-renewing cancer cells and tumor formation (Muhammad Al-Hajj and Michael F Clarke. Self-renewal and solid tumor stem cells Oncogene (2004) 23, 7274-7282). This suggests that agents that target the defective self-renewal pathways in cancer cells might lead to improved outcomes in the treatment of these diseases. Moreover, the CSCs grown in non-adherent cell suspension as tumorspheres showed better growth rate, increased stem cell marker CD44 expression and tumorigenicity when implanted in immunocompromised mice. Floating tumorsphere assays have become widely used to study in vitro cancer stem cell self-renewal and growth, a proxy for in vivo tumorigenesis.

Epithelial to mesenchymal transition (EMT) is a critical process for early stage carcinomas to become invasive malignancies. It is associated with the loss of epithelial phenotype and the gain of mesenchymal phenotype. Recent studies have demonstrated that EMT plays a critical role not only in tumor metastasis but also in tumor recurrence and is tightly linked with the biology of cancer stem cells. Recently it has been shown that cancer stem cells also emerge through the induction of EMT. The differentiated mammary epithelial cells that have undergone EMT either upon TGF-β treatment or by forced expression of E-cadherin transcriptional repressors, such as Snail, gave rise to CD44^{high}, CD24^{low} cells, akin to breast CSCs (Mani, S. A., Guo, W., Liao, M. J., Eaton, E. N., Ayyanan, A., Zhou, A. Y., Brooks, M., Reinhard, F., Zhang, C. C., Shipitsin, M., et al., 2008. The epithelial-mesenchymal transition generates cells with properties of stem cells. Cell. 2008; 133:704-715). The cancer stem cells generated from EMT induction provide a resource for cancer to reoccur. Moreover, these cells are well known to be highly drug resistant. Therefore, the molecular understanding and the biological characteristics of CSCs, such as cell growth by self-renewal and their EMT phenotype will allow us to screen for potential drugs that could cause selective killing of these cells thereby eradicating tumor recurrence.

Targeting cancer stem cells therapeutically directly or indirectly with Tissue Differentiation Factor Related Polypeptides (TDFRPs) may be useful in treating cancer.

### SUMMARY

The process of developing and administering a cell-based vaccine comprises two steps: 1) the harvesting of immune monocytes from a cancer patient which are processed ex vivo so as to differentiate them into immature dendritic cells (iDCs) and then to mature them into mature dendritic cells (mDCs); 2) introducing the mDCs, with or without TDFRPs, into cryo-ablated tumors of the cancer patient where the mDCs take up newly liberated foreign antigens and present them to immune T-cells as part of the T-cell training process.

The present disclosure describes improvements wherein: 1) a form of mDCs are differentiated and matured from monocytes in contact with one of more Tissue Differentiation Factor Related Polypeptides (TDFRPs) to produce mDCs that are more fully differentiated and matured, and are more stable; 2) the use of these mDCs in the presence of TDFRPs to enhance the efficacy of the in vivo T-cell training process; and 3) the use of systemically administered TDFRPs, to render cancer fibroblasts and cancer stem cells more sensitive to standard forms of therapeutic intervention.

According to one aspect, the present disclosure provides a method of producing mature dendritic cells, the method comprising: a) providing monocytes from a subject having cancer; b) contacting the monocytes with one or more tissue differentiation factor related polypeptides (TDFRPs), thereby producing immature dendritic cells; c) contacting the immature dendritic cells with one or more TDFRPs, thereby producing mature dendritic cells; and d) isolating mature dendritic cells.

In one embodiment, the monocytes are obtained from a subject having cancer.

In one embodiment, step b) further comprises contacting the monocytes with interleukin 4 (IL-4) and Granulocyte-macrophage colony-stimulating factor (GM-CSF).

In one embodiment, step c) further comprises contacting the immature dendritic cells with lipopolysaccharide.

In one embodiment, step b) comprises culturing the monocyes with one or more TDFRPs for about 3 to about 5 days. In one embodiment, the monocytes are cultured in step b) for about 3 days to about 5 days (e.g., 3 days, 3.5 day, 4 days, 4.5 days, 5 days).

In one embodiment, step c) comprises culturing the immature dendritic cells with one or more TDFRPs for about 24 hours to about 48 hours (e.g., 24 hours, 30 hours, 36 hours, 42 hours, 48 hours). In one embodiment, the immature dendritic cells are cultured in step c) for about 24 hours to about 48 hours (e.g., 24 hours, 30 hours, 36 hours, 42 hours, 48 hours).

In one embodiment, the one or more TDFRPs is selected from an amino acid sequence at least 90% identical to SEQ ID NO: 1 or SEQ ID NO:3. In one embodiment, the one or more TDFRPs is selected from an amino acid sequence at least 85% identical to SEQ ID NO: 1 or SEQ ID NO:3. In one embodiment, the one or more TDFRPs is selected from an amino acid sequence at least 90% identical to SEQ ID NO: 1 or SEQ ID NO:3. In one embodiment, the one or more TDFRPs is selected from an amino acid sequence at least 95% identical to SEQ ID NO: 1 or SEQ ID NO:3. In one embodiment, the one or more TDFRPs is selected from an amino acid sequence at least 96% identical to SEQ ID NO: 1 or SEQ ID NO:3. In one embodiment, the one or more TDFRPs is selected from an amino acid sequence at least 97% identical to SEQ ID NO: 1 or SEQ ID NO:3. In one embodiment, the one or more TDFRPs is selected from an amino acid sequence at least 98% identical to SEQ ID NO: 1 or SEQ ID NO:3. In one embodiment, the one or more TDFRPs is selected from an amino acid sequence at least 99% identical to SEQ ID NO: 1 or SEQ ID NO:3. In one embodiment, the one or more TDFRPs is selected from an amino acid sequence that comprises SEQ ID NO: 1 or SEQ ID NO:3. In one embodiment, the one or more TDFRPs is selected from an amino acid sequence consisting of SEQ ID NO: 1 or SEQ ID NO:3. In one embodiment, the one or more TDFRPs comprises amino acid sequence SEQ ID NO: 1. In one embodiment, the one or more TDFRPs comprises amino acid sequence SEQ ID NO: 3.

In some embodiments, the TDFRP comprises the peptide having the sequence of SEQ ID NO: 4. In some embodiments, the TDFRP comprises an amino acid sequence at least 85%, 90%, 95%, 96%, 97%, 98% or 99% identical to SEQ ID NO: 4. In some embodiments, the TDFPR comprises an amino acid sequence consisting of SEQ ID NO: 4.

In some embodiments, the TDFRP comprises the peptide having the sequence of SEQ ID NO: 5. In some embodiments, the TDFRP comprises an amino acid sequence at least 85%, 90%, 95%, 96%, 97%, 98% or 99% identical to SEQ ID NO: 5. In some embodiments, the TDFPR comprises an amino acid sequence consisting of SEQ ID NO: 5.

In one embodiment, the one or more TDFRPs comprises a crosslink.

In one embodiment, the one or more TDFRPs comprise a linker. In some embodiments, the linker is selected from a diamino alkane, a dicarboxylic acid, an amino carboxylic acid alkane, an amino acid sequence, e.g., glycine polypeptide, a disulfide linkage, a helical or sheet-like structural element or an alkyl chain.

In one embodiment, the cancer comprises cancer cells expressing bone morphogenetic protein (BMP) receptors.

In one embodiment, the cancer is breast cancer, prostate cancer, renal cell carcinoma, bone metastasis, lung cancer or metastasis, osteosarcoma, multiple myeloma, astrocytoma, pilocytic astrocytoma, dysembryoplastic neuroepithelial tumor, oligodendrogliomas, ependymoma, glioblastoma multiforme, mixed gliomas, oligoastrocytomas, medulloblastoma, retinoblastoma, neuroblastoma, germinoma, teratoma, gangliogliomas, gangliocytoma, central gangliocytoma, primitive neuroectodermal tumors (PNET, e.g. medulloblastoma, medulloepithelioma, neuroblastoma, retinoblastoma, ependymoblastoma), tumors of the pineal parenchyma (e.g. pineocytoma, pineoblastoma), ependymal cell tumors, choroid plexus tumors, neuroepithelial tumors of uncertain origin (e.g. gliomatosis cerebri, astroblastoma), esophageal cancer, colorectal cancer, CNS, ovarian, melanoma pancreatic cancer, squamous cell carcinoma, hematologic cancer (e.g., leukemia, lymphoma, and multiple myeloma), colon cancer, rectum cancer, stomach cancer, kidney cancer, mesothelioma, bladder cancer, skin cancer, or a combination thereof.

In one embodiment, the cancer is breast cancer.

In one embodiment, the cancer is a solid tumor. In one embodiment, the cancer is hematologic cancer.

According to one aspect, the present disclosure provides an isolated population of mature dendritic cells produced by the method of the above aspects or any other aspect of the invention delineated herein.

According to one aspect, the present disclosure provides an isolated population of mature dendritic cells, wherein the mature dendritic cells are differentiated from monocytes from a subject having cancer, and wherein the monocytes are contacted with one or more tissue differentiation factor related polypeptides (TDFRPs), thereby producing mature dendritic cells.

In one embodiment, the one or more TDFRPs comprises an amino acid sequence at least 85%, 90%, 95%, 96%, 97%, 98% or 99% identical to amino acid sequence SEQ ID NO: 1 or 3. In one embodiment, the one or more TDFRPs comprises SEQ ID NO: 1. In one embodiment, the one or more TDFRPs comprises SEQ ID NO: 3. In one embodiment, the one or more TDFRPs consists of SEQ ID NO: 1. In one embodiment, the one or more TDFRPs consists of SEQ ID NO: 3.

In some embodiments, the TDFRP comprises the peptide having the sequence of SEQ ID NO: 4. In some embodiments, the TDFRP comprises an amino acid sequence at least 85%, 90%, 95%, 96%, 97%, 98% or 99% identical to SEQ ID NO: 4. In some embodiments, the TDFPR comprises an amino acid sequence consisting of SEQ ID NO: 4.

In some embodiments, the TDFRP comprises the peptide having the sequence of SEQ ID NO: 5. In some embodiments, the TDFRP comprises an amino acid sequence at least 85%, 90%, 95%, 96%, 97%, 98% or 99% identical to SEQ ID NO: 5. In some embodiments, the TDFPR comprises an amino acid sequence consisting of SEQ ID NO: 5.

In one embodiment, the one or more TDFRPs comprises a crosslink.

In one embodiment, the one or more TDFRPs comprise a linker. In some embodiments, the linker is selected from a diamino alkane, a dicarboxylic acid, an amino carboxylic acid alkane, an amino acid sequence, e.g., glycine polypeptide, a disulfide linkage, a helical or sheet-like structural element or an alkyl chain

In one embodiment, the monocytes are first contacted with interleukin 4 (IL-4) and Granulocyte-macrophage colony-stimulating factor (GM-CSF) and the one or more TDFRPs, thereby producing immature dendritic cells.

In one embodiment, the monocytes are cultured for about 3 days to about 5 days (e.g., 3 days, 3.5 day, 4 days, 4.5 days, 5 days).

In one embodiment, the immature dendritic cells are contacted with lipopolysaccharide.

In one embodiment, the immature dendritic cells are cultured for about 24 hours to about 48 hours (e.g., 24 hours, 30 hours, 36 hours, 42 hours, 48 hours).

In one embodiment, the cancer comprises cancer cells expressing bone morphogenetic protein (BMP) receptors.

In one embodiment, the cancer is breast cancer, prostate cancer, renal cell carcinoma, bone metastasis, lung cancer or metastasis, osteosarcoma, multiple myeloma, astrocytoma, pilocytic astrocytoma, dysembryoplastic neuroepithelial tumor, oligodendrogliomas, ependymoma, glioblastoma multiforme, mixed gliomas, oligoastrocytomas, medulloblastoma, retinoblastoma, neuroblastoma, germinoma, teratoma, gangliogliomas, gangliocytoma, central gangliocytoma, primitive neuroectodermal tumors (PNET, e.g. medulloblastoma, medulloepithelioma, neuroblastoma, retinoblastoma, ependymoblastoma), tumors of the pineal parenchyma (e.g. pineocytoma, pineoblastoma), ependymal cell tumors, choroid plexus tumors, neuroepithelial tumors of uncertain origin (e.g. gliomatosis cerebri, astroblastoma), esophageal cancer, colorectal cancer, CNS, ovarian, melanoma pancreatic cancer, squamous cell carcinoma, hematologic cancer (e.g., leukemia, lymphoma, and multiple myeloma), colon cancer, rectum cancer, stomach cancer, kidney cancer, mesothelioma, bladder cancer, skin cancer, or a combination thereof. In one embodiment, the cancer is a solid tumor.

In one embodiment, the cancer is breast cancer.

According to one aspect, the present disclosure provides a method of treating a subject having cancer, comprising administering to the subject an effective amount of the isolated population of mature dendritic cells of any of the above aspects or any other aspect of the invention delineated herein.

In one embodiment, the method includes further administering one or more tissue differentiation factor related polypeptides (TDFRPs) to the subject. In one embodiment, a solid tumor was cryo-ablated prior to administration of the isolated population of mature dendritic cells and/or the one or more TDFRPs.

In one embodiment, the mature dendritic cells are administered to the same subject from which the monocytes were obtained. In one embodiment, the mature dendritic cells are administered to a different subject than the subject from which the monocytes were obtained.

In one embodiment, the cancer is breast cancer, prostate cancer, renal cell carcinoma, bone metastasis, lung cancer or metastasis, osteosarcoma, multiple myeloma, astrocytoma, pilocytic astrocytoma, dysembryoplastic neuroepithelial tumor, oligodendrogliomas, ependymoma, glioblastoma multiforme, mixed gliomas, oligoastrocytomas, medulloblastoma, retinoblastoma, neuroblastoma, germinoma, teratoma, gangliogliomas, gangliocytoma, central gangliocytoma, primitive neuroectodermal tumors (PNET, e.g., medulloblastoma, medulloepithelioma, neuroblastoma, retinoblastoma, ependymoblastoma), tumors of the pineal parenchyma (e.g., pineocytoma, pineoblastoma), ependymal cell tumors, choroid plexus tumors, neuroepithelial tumors of uncertain origin (e.g. gliomatosis cerebri, astroblastoma), esophageal cancer, colorectal cancer, CNS, ovarian, melanoma pancreatic cancer, squamous cell carcinoma, hematologic cancer (e.g., leukemia, lymphoma, and multiple myeloma), colon cancer, rectum cancer, stomach cancer, kidney cancer, mesothelioma, bladder cancer, skin cancer, or a combination thereof.

In one embodiment, the cancer is breast cancer.

According to one aspect, the disclosure provides a method of treating a subject having a chemoresistant cancer, the method comprising administering one or more tissue differentiation factor related polypeptides (TDFRPs) to the subject. According to some embodiments, the chemoresistant cancer is selected from a chemoresistant breast cancer, a chemoresistant ovarian cancer, a chemoresistant prostate cancer, a chemoresistant lung cancer and a chemoresistant colorectal cancer. According to some embodiments, the chemoresistant cancer is metastatic.

According to another aspect, the disclosure provides a method of delaying the progression of a cancer in a subject, the method comprising administering one or more tissue differentiation factor related polypeptides (TDFRPs) to the subject, wherein administration of one or more TDFRPs reduces or stabilizes tumor growth over time compared to a control.

According to one aspect, the present disclosure provides a method of treating a subject having cancer, the method comprising administering one or more tissue differentiation factor related polypeptides (TDFRPs) to the subject, thereby rendering a cancer cell responsive to one or more antineoplastic modalities, and thereby treating the subject. In one embodiment, the subject is being treated with the antineoplastic modality and the TDFRP concurrently. In one embodiment, the subject is administered the antineoplastic modality before or after, or both before and after, administration of the TDFRP.

In one embodiment, the cancer cell expresses one or more epithelial markers after treatment with the one or more TDFRPs. In a further embodiment, the epithelial marker is E-cadherin.

In one embodiment of any of the aspects and embodiments herein, the one or more TDFRPs comprises an amino acid sequence at least 90%, 95%, 96%, 97%, 98% or 99% identical to amino acid sequence SEQ ID NO: 1 or 3. In one embodiment, the one or more TDFRPs comprises SEQ ID NO: 1. In one embodiment, the one or more TDFRPs comprises SEQ ID NO: 3. In one embodiment, the one or more TDFRPs consists of SEQ ID NO: 1. In one embodiment, the one or more TDFRPs consists of SEQ ID NO: 3. In one embodiment, the one or more TDFRPs comprises a crosslink.

In one embodiment, the cancer cells express bone morphogenetic protein (BMP) receptors.

In one embodiment, the cancer is breast cancer, prostate cancer, renal cell carcinoma, bone metastasis, lung cancer or metastasis, osteosarcoma, multiple myeloma, astrocytoma, pilocytic astrocytoma, dysembryoplastic neuroepithelial tumor, oligodendrogliomas, ependymoma, glioblastoma multiforme, mixed gliomas, oligoastrocytomas, medulloblastoma, retinoblastoma, neuroblastoma, germinoma, teratoma, gangliogliomas, gangliocytoma, central gangliocytoma, primitive neuroectodermal tumors (PNET, e.g. medulloblastoma, medulloepithelioma, neuroblastoma, retinoblastoma, ependymoblastoma), tumors of the pineal parenchyma (e.g. pineocytoma, pineoblastoma), ependymal cell tumors, choroid plexus tumors, neuroepithelial tumors of uncertain origin (e.g. gliomatosis cerebri, astroblastoma), esophageal cancer, colorectal cancer, CNS, ovarian, melanoma pancreatic cancer, squamous cell carcinoma, hematologic cancer (e.g., leukemia, lymphoma, and multiple myeloma), colon cancer, rectum cancer, stomach cancer, kidney cancer, mesothelioma, bladder cancer, skin cancer, or a combination thereof.

In one embodiment, the cancer is breast cancer.

In one embodiment, the cancer is a solid tumor.

In one embodiment, the cancer cells are resistant to the one or more antineoplastic modalities. In one embodiment, the cancer cells are resistant to one or more antineoplastic modalities other than one or more antineoplastic modalities of the above aspects or any other aspect of the invention delineated herein.

In one embodiment, the one or more antineoplastic modalities are one or more chemotherapeutic agents, surgery, radiation, immunotherapy, hormone therapy, stem cell transplant, small-molecules, antibodies, chimeric antigen receptor T cells (CAR-T cells), cancer vaccines, or a combination thereof.

According to one aspect, the present disclosure provides an in vivo method of increasing T-cell recognition of tumor cell antigens on tumor cells in a subject having cancer, comprising administering to the subject an effective amount of the isolated population of mature dendritic cells of the above aspects or any other aspect of the invention delineated herein, wherein TGF-beta secretion from the tumor cells is decreased compared to a control tumor cell.

### BRIEF DESCRIPTION OF DRAWINGS

FIG. 1 shows loss of Mammospheres after Peptide 123 or BMP-7 Treatment. Isolated human breast cancer stem cells grown in stem cell culture medium alone showed efficient mammosphere formation after 5 days in non-adherent culture. Cells treated with P123 (300 μM) showed profound inhibition of mammosphere formation. Cells treated with BMP-7 (500 ng/ml) showed moderate inhibition of mammosphere formation.

FIG. 2 shows recovery of Mammospheres after Withdrawal of BMP-7. Isolated human breast cancer stem cells treated with BMP-7 (500 ng/ml) for 7 days in non-adherent culture. BMP-7 treatment was discontinued and after 7 days with no treatment, cells showed significant increase in Mammosphere formation.

FIG. 3 shows FACS analysis of untreated cells vs. corresponding isotype control. Untreated cells were incubated with APC labeled mouse anti-human antibody to CD44 antibody and FITC labeled rat anti-human E-Cadherin antibody. Cells were gated to exclude events in water and events due to cell death and debris. Cells were then gated to identify CD44+ and E-Cadherin+ populations based on corresponding fluorescence activity and labeled R7 and R11 respectively. FACS analysis identified CD44+ cells as seen in R7 and E-cadherin+ cells as seen in R11. Untreated cells were incubated with corresponding isotype antibodies, APC labeled mouse IgG1 and FITC labeled mouse IgG1. Cells were gated to exclude events in water and events due to cell death and debris. FACS analysis showed little background fluorescence due to Fc or other non-specific antibody binding as indicated by low activity in R7 and R11.

FIG. 4 shows FACS analysis of peptide treated cells vs. corresponding isotype control. Cells were plated and incubated for 24 hours to form tumorspheres. On day 2, cells were treated with 300 μM P123, followed by subsequent treatments on days 3, 5, and 8. Cells were then incubated with APC labeled mouse anti-human CD44 antibody and FITC labeled rat anti-human E-Cadherin antibody. Cells were gated to exclude events in water and events due to cell death and debris. Cells were then gated to identify CD44+ and E-Cadherin+ populations based on corresponding fluorescence activity and labeled R7 and R11 respectively. FACS analysis identified CD44+ cells as seen in R7 and E-cadherin+ cells as seen in R11. Cells were treated with P123 as described in (A). Cells were then incubated with corresponding isotype antibodies, APC labeled mouse IgG1 and FITC labeled mouse IgG1. Cells were gated to exclude events in water and events due to cell death and debris. FACS analysis initially showed little background fluorescence due to Fc or other non-specific antibody binding as indicated by low activity in R7 and R11, however during testing, an air bubble was aspirated producing the slight background observed.

FIG. 5 shows FACS analysis of BMP-7 treated cells vs. corresponding isotype control. Cells were plated and incubated for 24 hours to form tumorspheres. On day 2, cells were treated with BMP-7 at 430 ng/ml with subsequent treatments at 362 ng/ml on day 3, 314 ng/ml on day 5, and 277 ng/ml on day 8 (media was added at 10% volume resulting in decreased concentrations). Cells were then incubated with APC labeled mouse anti-human CD44 antibody and FITC labeled rat anti-human E-Cadherin antibody. Cells were gated to exclude events in water and events due to cell death and debris. Cells were then gated to identify CD44+ and E-Cadherin+ populations based on corresponding fluorescence activity and labeled R7 and R11 respectively. FACS analysis identified CD44+ cells as seen in R7 and E-cadherin+ cells as seen in R11. Cells were treated with BMP-7 as described in (A). Cells were then incubated with corresponding isotype antibodies, APC labeled mouse IgG1 and FITC labeled mouse IgG1. Cells were gated to exclude events in water and events due to cell death and debris. FACS analysis showed little background fluorescence due to Fc or other non-specific antibody binding as indicated by low activity in R7 and R11.

FIG. 6 shows loss of CD44+ and gain of E-Cadherin+ cells after P123 or BMP-7 treatment. FACS analysis was performed to quantify CD44+ and E-Cadherin+ cells in no treatment, P123 treated, and BMP-7 treated stem cells. In comparison to control, P123 treatment resulted in marked decrease in CD44+ cells and marked increase in E-Cadherin+ cells. This suggests that P123 may have caused loss of stem cell phenotype and acquisition of epithelial phenotype. Similarly, in comparison to control, BMP-7 treatment showed a decrease in CD44+ cells and an increase in E-Cadherin cells. This suggests that BMP-7 may have also caused loss of stem cell phenotype and gain of epithelial phenotype in some cells. Overall, corresponding isotype controls indicate little background due to non-specific fluorescence.

FIG. 7 shows loss of CD44+ and E-Cadherin+ cells as percentage of total cells. FACS analysis was performed to quantify CD44+ and E-Cadherin+ cells in no treatment, P123 treated, and BMP-7 treated stem cells. The majority of cancer stem cells are CD44+, with a minority representing MCF-7 cells from which these CSC's were isolated. In comparison to control, the majority of P123 treated cells were E-Cadherin+, indicating a transition from stem cell phenotype to epithelial phenotype. Similarly, but to a lesser extent, BMP-7 treated cells showed an increase E-Cadherin+ with a large portion of CD44+ remaining. This suggests some cells undergoing transition to epithelial phenotype with a large number of cells that were unaffected.

### DESCRIPTION

Provided herein are TDFRPs that can advantageously be used to treat or ameliorate cancer in a subject.

### Definitions

As used in this specification and the appended claims, the singular forms “a”, “an” and “the” include plural references unless the content clearly dictates otherwise.

The use of the alternative (e.g., “or”) should be understood to mean either one, both, or any combination thereof of the alternatives.

As used herein, the term “about,” when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

As used herein, any concentration range, percentage range, ratio range, or integer range is to be understood to include the value of any integer within the recited range and, when appropriate, fractions thereof (such as one tenth and one hundredth of an integer), unless otherwise indicated.

As used herein, “comprise,” “comprising,” and “comprises” and “comprised of” are meant to be synonymous with “include”, “including”, “includes” or “contain”, “containing”, “contains” and are inclusive or open-ended terms that specifies the presence of what follows e.g. component and do not exclude or preclude the presence of additional, non-recited components, features, element, members, steps, known in the art or disclosed therein.

As used herein, the terms “such as”, “for example” and the like are intended to refer to exemplary embodiments and not to limit the scope of the present disclosure.

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, preferred materials and methods are described herein.

As used herein, “administration,” “administering” and variants thereof refers to introducing a composition or agent into a subject and includes concurrent and sequential introduction of a composition or agent. “Administration” can refer, e.g., to therapeutic, pharmacokinetic, diagnostic, research, placebo, and experimental methods. “Administration” also encompasses in vitro and ex vivo treatments. The introduction of a composition or agent into a subject is by any suitable route, including orally, pulmonarily, intranasally, parenterally (intravenously, intramuscularly, intraperitoneally, or subcutaneously), rectally, intralymphatically, or topically. Administration includes self-administration and the administration by another. Administration can be carried out by any suitable route. A suitable route of administration allows the composition or the agent to perform its intended function. For example, if a suitable route is intravenous, the composition is administered by introducing the composition or agent into a vein of the subject. According to some embodiments, the pharmaceutical compositions disclosure herein can be administered intravenously, intratumorally, subcutaneously, or intramuscularly. According to some embodiments, the pharmaceutical compositions are administered intratumorally.

As used herein, the term “analog” is meant to refer to a composition that differs from the compound of the present disclosure but retains essential properties thereof. A non-limiting example of this is a polypeptide or peptide or peptide fragment that includes non-natural amino acids, peptidomimetics, unusual amino acids, amide bond isosteres.

As used herein, the term “cancer” refers to diseases in which abnormal cells divide without control and are able to invade other tissues. There are more than 100 different types of cancer. Most cancers are named for the organ or type of cell in which they start-for example, cancer that begins in the colon is called colon cancer; cancer that begins in melanocytes of the skin is called melanoma. Cancer types can be grouped into broader categories. The main categories of cancer include: carcinoma (meaning a cancer that begins in the skin or in tissues that line or cover internal organs, and its subtypes, including adenocarcinoma, basal cell carcinoma, squamous cell carcinoma, and transitional cell carcinoma); sarcoma (meaning a cancer that begins in bone, cartilage, fat, muscle, blood vessels, or other connective or supportive tissue); leukemia (meaning a cancer that starts in blood-forming tissue (e.g., bone marrow) and causes large numbers of abnormal blood cells to be produced and enter the blood; lymphoma and myeloma (meaning cancers that begin in the cells of the immune system); and central nervous system (CNS) cancers (meaning cancers that begin in the tissues of the brain and spinal cord). In certain embodiments, the cancer is selected from cancers including, but not limited to, ACUTE lymphoblastic leukemia (ALL), ACUTE myeloid leukemia (AML), anal cancer, bile duct cancer, bladder cancer, bone cancer, bowel cancer, brain tumor, breast cancer, cancer of unknown primary, cancer spread to bone, cancer spread to brain, cancer spread to liver, cancer spread to lung, carcinoid, cervical cancer, choriocarcinoma, chronic lymphocytic leukemia (CLL), chronic myeloid leukemia (CML), colon cancer, colorectal cancer, endometrial cancer, eye cancer, gallbladder cancer, gastric cancer, gestational trophoblastic tumor (GTT), hairy cell leukemia, head and neck cancer, Hodgkin lymphoma, kidney cancer, laryngeal cancer, leukemia, liver cancer, lung cancer, lymphoma, melanoma skin cancer, mesothelioma, men's cancer, molar pregnancy, mouth and oropharyngeal cancer, myeloma, nasal and sinus cancers, nasopharyngeal cancer, non Hodgkin lymphoma (NHL), esophageal cancer, ovarian cancer, pancreatic cancer, penile cancer, prostate cancer, rare cancers, rectal cancer, salivary gland cancer, secondary cancers, skin cancer (non melanoma), soft tissue sarcoma, stomach cancer, testicular cancer, thyroid cancer, unknown primary cancer, uterine cancer, vaginal cancer, and vulvar cancer. According to one embodiment, the cancer is prostate cancer. According to one embodiment, the cancer is breast cancer.

As used herein, the term “dendritic cell” or “DC”, includes immature and mature dendritic cells and related myeloid progenitor cells that are capable of differentiating into dendritic cells, or related antigen presenting cells (e.g., monocytes and macrophages) in that they express antigens in common with dendritic cells. As used herein, the term “related” includes a cell that is derived from a common progenitor cell or cell lineage. In a preferred embodiment, binding of an antibody of the invention to a dendritic cell inhibits the growth of dendritic cells. In another preferred embodiment, binding of an antibody of the invention to dendritic cells mediates an effect on dendritic cell growth and/or function by targeting molecules or cells with defined functions (e.g., tumor cells, effector cells, microbial pathogens) to dendritic cells. In a further embodiment, binding of an antibody of the invention to a dendritic cell results in internalization of the antibody by the dendritic cell.

Activation of dendritic cells initiates the process that converts immature DCs, which are phenotypically similar to skin Langerhans cells, to mature, antigen presenting cells that can migrate to the lymph nodes. This process results in the gradual and progressive loss of the powerful antigen uptake capacity that characterizes the immature dendritic cell, and in the up-regulation of expression of co-stimulatory cell surface molecules and various cytokines. Various stimuli can initiate the maturation of DCs. This process is complex and at least in vitro can take up to 48 hours to complete. One other consequence of maturation is a change in the in vivo migratory properties of the cells. For example, maturation induces several chemokine receptors, including CCR7, which direct the cells to the T cell regions of draining lymph nodes, where the mature DCs activate T cells against the antigens presented on the DC surface in the context of class I and class II MHC molecules. The terms “activation” and “maturation”, and “activated” and “mature” describe the process of inducing and completing the transition from an immature DC (partially characterized by the ability to take up antigen) to a mature DC (partially characterized by the ability to effectively stimulate de novo T cell responses). The terms typically are used interchangeably in the art.

As used herein, an “effective amount” of a compound is meant to refer to a quantity sufficient to achieve a desired therapeutic and/or prophylactic effect, for example, an amount which results in the prevention of or a decrease in the symptoms associated with a disease that is being treated, e.g., cancer. The amount of compound administered to the subject will depend on the type and severity of the disease and on the characteristics of the individual, such as general health, age, sex, body weight and tolerance to drugs. It will also depend on the degree, severity and type of disease. The skilled artisan will be able to determine appropriate dosages depending on these and other factors. Typically, an effective amount of the compounds of the present disclosure, sufficient for achieving a therapeutic or prophylactic effect, range from about 0.000001 mg per kilogram body weight per day, to about 10,000 mg per kilogram body weight per day. Preferably, the dosage ranges are from about 0.0001 mg per kilogram body weight per day to about 100 mg per kilogram body weight per day. The compounds of the present disclosure can also be administered in combination with each other, or with one or more additional therapeutic compounds.

As used herein, an “isolated” or “purified” polypeptide or polypeptide or biologically-active portion thereof is substantially free of cellular material or other contaminating polypeptides from the cell or tissue source from which the tissue differentiation factor-related polypeptide is derived, or substantially free from chemical precursors or other chemicals when chemically synthesized.

As used herein, the term “monocytes” refers to a particular type of white blood cells (leukocytes) that are part of the innate immune system of vertebrates. Monocytes are normally produced by the bone marrow from hematopoietic stem cell precursors called monoblasts. They circulate in the bloodstream for about one to three days and then typically move into tissues throughout the body. Monocytes constitute between three to eight percent of the leukocytes in the blood (reference values in healthy adult humans), and are the largest corpuscle in blood. Once extravasated from the bloodstream to other tissues, they will differentiate into tissue resident macrophages or dendritic cells. Monocytes play multiple roles in immune function such as: (1) replenishing resident macrophages under normal states, and (2) rapidly migrating to sites of infection or metabolic stress in tissues in response to inflammation signals. There are at least three types of monocytes in human blood: a) the classical monocyte, which is characterized by high level expression of the CD14 cell surface receptor (CD14++CD16− monocyte), b) the non-classical monocyte, which shows low level expression of CD14 and high level expression of the CD16 receptor (CD14+CD16++ monocyte), and c) the intermediate monocyte with high level expression of CD14 and low level expression of CD16 (CD14++CD16+ monocytes). In humans, CD14 is considered a marker of the monocyte lineage. So, at least in humans, “monocytes” can be considered equivalent to CD14-expressing cells that circulate in the bloodstream (the latter property distinguishing them from dendritic cells and macrophages). Although virtually all CD14-expressing cells in peripheral blood will be monocytes, further differentiation using other markers or cell size can be made to distinguish monocytes from other cell types.

As used herein, the terms “polypeptide”, “peptide” and “protein” are used interchangeably herein to refer to a natural or synthetic peptide containing two or more amino acids linked typically via the carboxy group of one amino acid and the amino group of another amino acid. As will be appreciated by those having skill in the art, the above definition is not absolute and polypeptides or peptides can include other examples where one or more amide bonds could be replaced by other bonds, for example, isosteric amide bonds. The terms apply to amino acid polymers in which one or more amino acid residue is an artificial chemical analogue of a corresponding naturally occurring amino acid, as well as to naturally occurring amino acid polymers. The essential nature of such analogues of naturally occurring amino acids is that, when incorporated into a protein, that protein is specifically reactive to antibodies elicited to the same protein but consisting entirely of naturally occurring amino acids. The terms “polypeptide”, “peptide” and “protein” also are inclusive of modifications including, but not limited to, glycosylation, lipid attachment, sulfation, gamma-carboxylation of glutamic acid residues, hydroxylation, and ADP-ribosylation. It will be appreciated, as is well known and as noted above, that polypeptides may not be entirely linear. For instance, polypeptides may be branched as a result of ubiquitination, and they may be circular, with or without branching, generally as a result of posttranslational events, including natural processing event and events brought about by human manipulation which do not occur naturally. Circular, branched and branched circular polypeptides may be synthesized by non-translation natural process and by entirely synthetic methods, as well.

As used herein, the term “pharmaceutically acceptable carrier” includes any of the standard pharmaceutical carriers, such as a phosphate buffered saline solution, water, emulsions such as an oil/water or water/oil, and various types of wetting agents. The term also encompasses any of the agents approved by a regulatory agency of the US Federal government or listed in the US Pharmacopeia for use in animals, including humans, as well as any carrier or diluent that does not cause significant irritation to a subject and does not abrogate the biological activity and properties of the administered compound.

As used herein, the term “small molecule” is meant to refer to a composition that has a molecular weight of less than about 5 kDa and more preferably less than about 2 kDa. Small molecules can be, e.g., nucleic acids, peptides, polypeptides, glycopeptides, peptidomimetics, carbohydrates, lipids, lipopolysaccharides, combinations of these, or other organic or inorganic molecules.

As used herein, the terms “subject,” “individual,” “host,” and “patient,” are used interchangeably herein and refer to any mammalian subject for whom diagnosis, treatment, or therapy is desired, particularly humans. The methods described herein are applicable to both human therapy and veterinary applications. According to some embodiments, the subject is a mammal, and in other embodiments the subject is a human. According to some embodiments, a “subject in need” is meant to refer to a subject that has been diagnosed with cancer. According to some embodiments, a “subject in need” is meant to refer to a subject that is susceptible to caner. Non-limiting examples of a subject that is susceptible to cancer include genetically predisposed subjects or elderly subjects.

As used herein, the terms “therapeutic amount”, “therapeutically effective amount”, an “amount effective”, or “pharmaceutically effective amount” of an active agent (e.g. a TDFRP), as described herein, are used interchangeably to refer to an amount that is sufficient to provide the intended benefit of treatment (e.g., reduction of tumor size, remission of symptoms of cancer). However, dosage levels are based on a variety of factors, including the type of injury, the age, weight, sex, medical condition of the patient, the severity of the condition, the route of administration, and the particular active agent employed. Thus the dosage regimen may vary widely, but can be determined routinely by a physician using standard methods. Additionally, the terms “therapeutic amount”, “therapeutically effective amounts” and “pharmaceutically effective amounts” include prophylactic or preventative amounts of the compositions of the disclosure. In prophylactic or preventative applications of the disclosure, pharmaceutical compositions or medicaments are administered to a patient susceptible to, or otherwise at risk of, a disease, disorder or condition in an amount sufficient to eliminate or reduce the risk, lessen the severity, or delay the onset of the disease, disorder or condition, including biochemical, histologic and/or behavioral symptoms of the disease, disorder or condition, its complications, and intermediate pathological phenotypes presenting during development of the disease, disorder or condition. It is generally preferred that a maximum dose be used, that is, the highest safe dose according to some medical judgment. The terms “dose” and “dosage” are used interchangeably herein.

As used herein “therapeutically responsive” or “responsive” is meant that a subject suffering from the disease or disorder will enjoy at least one of the following clinical benefits as a result of treatment with one or more TDFRPs and mature dendritic cells: amelioration of the disease or disorder, reduction in the occurrence of symptoms associated with the disease or disorder, partial remission of the disease or disorder, full remission of the disease or disorder, or increased time to progression. In other words, the therapeutic response can be a full or partial therapeutic response, and the method is used to determine the probability of a therapeutic response, regardless of whether it is a full or partial response.

It should be noted that a therapeutic response can be a full or partial response at therapeutically relevant doses to a subject according to a prescribed dosing regimen. In other words, the level of predicted therapeutic response is determined at a dose that would not be fatal to a subject to which the pharmaceutical composition, e.g., one or more TDFRPs and mature dendritic cells, is administered. A therapeutically relevant dose, therefore, is a therapeutic dose according to a prescribed dosing regimen at which a therapeutic response of the disease or disorder to treatment with a pharmaceutical composition, e.g., one or more TDFRPs and mature dendritic cells, is observed and at which a subject can be administered the pharmaceutical composition, e.g., one or more TDFRPs and mature dendritic cells, without an excessive amount of unwanted or deleterious side effects. A therapeutically relevant dose is non-lethal to a subject, even though it may cause some side effects in the patient. It is a dose at which the level of clinical benefit to a subject being administered the pharmaceutical composition, e.g., one or more TDFRPs and mature dendritic cells, exceeds the level of deleterious side effects experienced by the subject due to administration of the pharmaceutical composition, e.g., one or more TDFRPs and mature dendritic cells.

As used herein, the term “Transforming Growth Factor-beta (TGF-β) superfamily of polypeptides,” is meant to refer to a superfamily of polypeptide factors with pleiotropic functions that is composed of many multifunctional cytokines which includes, but is not limited to, TGF-ßs, activins, inhibins, anti-müllerian hormone (AMH), mullerian inhibiting substance (MIS), bone morphogenetic proteins (BMPs), and myostatin. The highly similar TGF-ß isoforms TGF-ß1, TGF-ß2, and TGF-ß3 potently inhibit cellular proliferation of many cell types, including those from epithelial origin. Most mesenchymal cells, however, are stimulated in their growth by TGF-ß. In addition, TGF-ßs strongly induce extracellular matrix synthesis and integrin expression, and modulate immune responses. BMPs, also known as osteogenic proteins (OPs), are potent inducers of bone and cartilage formation and play important developmental roles in the induction of ventral mesoderm, differentiation of neural tissue, and organogenesis. Activins, named after their initial identification as activators of follicle-stimulating hormone (FSH) secretion from pituitary glands, are also known to promote erythropoiesis, mediate dorsal mesoderm induction, and contribute to survival of nerve cells. Several growth factors belonging to the TGF-ß superfamily play important roles in embryonic patterning and tissue homeostasis. Their inappropriate functioning has been implicated in several pathological situations like cancer, e.g., prostate cancer. The term, tissue differentiation factor (TDF), as used herein, includes, but is not limited to, all members of the TGF-beta superfamily of polypeptides. TGF-beta superfamily polypeptides can be antagonists or agonists of TGF-beta superfamily receptors.

As used herein, the term “Transforming Growth Factor-beta (TGF-beta) superfamily receptors,” is meant to refer to polypeptide receptors that mediate the pleiotropic effects of transforming growth factor-ß (TGF-ß) superfamily polypeptides, as well as fragments, analogs and homologs thereof. Such receptors may include, but are not limited to, distinct combinations of Type I and Type II serine/threonine kinase receptors. The term, tissue differentiation factor receptor (TDF), as used herein, includes, but is not limited to, all members of the TGF-beta superfamily of receptors.

As used herein, the terms “treat,” “treating,” and/or “treatment” include abrogating, substantially inhibiting, slowing or reversing the progression of a condition, substantially ameliorating clinical symptoms of a condition, or substantially preventing the appearance of clinical symptoms of a condition, obtaining beneficial or desired clinical results. Treating further refers to accomplishing one or more of the following: (a) reducing the severity of the disorder; (b) limiting development of symptoms characteristic of the disorder(s) being treated; (c) limiting worsening of symptoms characteristic of the disorder(s) being treated; (d) limiting recurrence of the disorder(s) in patients that have previously had the disorder(s); and (e) limiting recurrence of symptoms in patients that were previously asymptomatic for the disorder(s).

Beneficial or desired clinical results, such as pharmacologic and/or physiologic effects include, but are not limited to, preventing the disease, disorder or condition from occurring in a subject that may be predisposed to the disease, disorder or condition but does not yet experience or exhibit symptoms of the disease (prophylactic treatment), alleviation of symptoms of the disease, disorder or condition, diminishment of extent of the disease, disorder or condition, stabilization (i.e., not worsening) of the disease, disorder or condition, preventing spread of the disease, disorder or condition, delaying or slowing of the disease, disorder or condition progression, amelioration or palliation of the disease, disorder or condition, and combinations thereof, as well as prolonging survival as compared to expected survival if not receiving treatment.

As used herein the term “therapeutic effect” refers to a consequence of treatment, the results of which are judged to be desirable and beneficial. A therapeutic effect can include, directly or indirectly, the arrest, reduction, or elimination of a disease manifestation. A therapeutic effect can also include, directly or indirectly, the arrest reduction or elimination of the progression of a disease manifestation.

For any therapeutic agent described herein the therapeutically effective amount may be initially determined from preliminary in vitro studies and/or animal models. A therapeutically effective dose may also be determined from human data. The applied dose may be adjusted based on the relative bioavailability and potency of the administered compound. Adjusting the dose to achieve maximal efficacy based on the methods described above and other well-known methods is within the capabilities of the ordinarily skilled artisan. General principles for determining therapeutic effectiveness, which may be found in Chapter 1 of Goodman and Gilman's The Pharmacological Basis of Therapeutics, 10th Edition, McGraw-Hill (New York) (2001), incorporated herein by reference, are summarized below.

Pharmacokinetic principles provide a basis for modifying a dosage regimen to obtain a desired degree of therapeutic efficacy with a minimum of unacceptable adverse effects. In situations where the drug's plasma concentration can be measured and related to the therapeutic window, additional guidance for dosage modification can be obtained.

Drug products are considered to be pharmaceutical equivalents if they contain the same active ingredients and are identical in strength or concentration, dosage form, and route of administration. Two pharmaceutically equivalent drug products are considered to be bioequivalent when the rates and extents of bioavailability of the active ingredient in the two products are not significantly different under suitable test conditions.

As used herein, the term “variant,” is meant to refer to a compound that differs from the compound of the present disclosure, but retains essential properties thereof. A non-limiting example of this is a polynucleotide or polypeptide compound having conservative substitutions with respect to the reference compound, commonly known as degenerate variants. Another non-limiting example of a variant is a compound that is structurally different, but retains the same active domain of the compounds of the present disclosure. Variants include N-terminal or C-terminal extensions, capped amino acids, modifications of reactive amino acid side chain functional groups, e.g., branching from lysine residues, pegylation, and/or truncations of a polypeptide compound. Generally, variants are overall closely similar, and in many regions, identical to the compounds of the present disclosure. Accordingly, the variants may contain alterations in the coding regions, non-coding regions, or both.

### Compositions

According to one aspect, the present disclosure provides compositions comprising mature dendritic cells and one or more TDFRPs. The present disclosure provides in another aspect methods of producing mature dendritic cells. The present disclosure provides in another aspect methods of treating a subject having cancer with a composition comprising mature dendritic cells and one or more TDFRPs. The present disclosure provides in another aspect method of treating a subject having cancer with mature dendritic cells and one or more TDFRPs, thereby rendering a cancer cell responsive to one or more antineoplastic modalities. The present disclosure provides in another aspect methods of increasing T-cell recognition of tumor cell antigens by administering mature dendritic cells and one or more TDFRPs, thereby decreasing TGF-beta secretion from the cancer cell compared to a control cancer cell.

### Tissue Differentiation Factor Related Polypeptides (TDFRPs)

The present disclosure provides compounds that are functional analogs of tissue differentiation factors, i.e., compounds that functionally mimic TGF-beta superfamily proteins, for example by acting as TGF-beta superfamily receptor agonists, and preferentially bind to select ALK receptor(s). The present compounds are called TDFRPs, and include small molecules, more particularly polypeptides.

According to one embodiment, the TDFRP compound has the general structure identified as SEQ ID NOs:1-208, disclosed in International Publication No. WO/2003/106656, incorporated by reference in its entirety herein. According to one embodiment, a TDFRP compound includes an analog or homolog of SEQ ID NOs:1-208. Compounds of the present disclosure include those with homology to SEQ ID NOs:1-208, for example, preferably 50% or greater amino acid identity, more preferably 75% or greater amino acid identity, and even more preferably 90% or greater amino acid identity. The compounds of the present disclosure also include one or more polynucleotides encoding SEQ ID NOs:1-208, including degenerate variants thereof. Accordingly, nucleic acid sequences capable of hybridizing at low stringency with any nucleic acid sequences encoding SEQ ID NOs:1-208 are considered to be within the scope of the disclosure.

According to one embodiment, the TDFRP compound has the general structure identified as SEQ ID NOs:1-347, disclosed in International Publication No. WO/2007/035872, incorporated by reference in its entirety herein. According to one embodiment, a TDFRP compound includes an analog or homolog of SEQ ID NOs:1-347. Compounds of the present disclosure include those with homology to SEQ ID NOs:1-347, for example, preferably 50% or greater amino acid identity, more preferably 75% or greater amino acid identity, and even more preferably 90% or greater amino acid identity. The compounds of the present disclosure also include one or more polynucleotides encoding SEQ ID NOs:1-347, including degenerate variants thereof. Accordingly, nucleic acid sequences capable of hybridizing at low stringency with any nucleic acid sequences encoding SEQ ID NOs:1-347 are considered to be within the scope of the disclosure.

According to one embodiment, the TDFRP compound has the general structure identified as SEQ ID NOs:1-314, disclosed in International Publication No. WO/2006/009836, incorporated by reference in its entirety herein. According to one embodiment, a TDFRP compound includes an analog or homolog of SEQ ID NOs:1-314. Compounds of the present disclosure include those with homology to SEQ ID Nos:1-314, for example, preferably 50% or greater amino acid identity, more preferably 75% or greater amino acid identity, and even more preferably 90% or greater amino acid identity. The compounds of the present disclosure also include one or more polynucleotides encoding SEQ ID NOs:1-314, including degenerate variants thereof. Accordingly, nucleic acid sequences capable of hybridizing at low stringency with any nucleic acid sequences encoding SEQ ID NOs:1-314 are considered to be within the scope of the disclosure.

Sequence identity can be measured using sequence analysis software (Sequence Analysis Software Package of the Genetics Computer Group, University of Wisconsin Biotechnology Center, 1710 University Avenue, Madison, Wis. 53705), with the default parameters therein.

In the case of polypeptide sequences, which are less than 100% identical to a reference sequence, the non-identical positions are preferably, but not necessarily, conservative substitutions for the reference sequence. Conservative substitutions typically include substitutions within the following groups: glycine and alanine; valine, isoleucine, and leucine; aspartic acid and glutamic acid; asparagine and glutamine; serine and threonine; lysine and arginine; and phenylalanine and tyrosine. Thus, included in the disclosure are peptides having mutated sequences such that they remain homologous, e.g., in sequence, in structure, in function, and in antigenic character or other function, with a polypeptide having the corresponding parent sequence. Such mutations can, for example, be mutations involving conservative amino acid changes, e.g., changes between amino acids of broadly similar molecular properties. For example, interchanges within the aliphatic group alanine, valine, leucine and isoleucine can be considered as conservative. Sometimes substitution of glycine for one of these can also be considered conservative. Other conservative interchanges include those within the aliphatic group aspartate and glutamate; within the amide group asparagine and glutamine; within the hydroxyl group serine and threonine; within the aromatic group phenylalanine, tyrosine and tryptophan; within the basic group lysine, arginine and histidine; and within the sulfur-containing group methionine and cysteine. Sometimes substitution within the group methionine and leucine can also be considered conservative. Preferred conservative substitution groups are aspartate-glutamate; asparagine-glutamine; valine-leucine-isoleucine; alanine-valine; phenylalanine-tyrosine; and lysine-arginine.

The disclosure also provides for compounds having altered sequences including insertions such that the overall amino acid sequence is lengthened, while the compound still retains the appropriate TDF agonist or antagonist properties. Additionally, altered sequences may include random or designed internal deletions that truncate the overall amino acid sequence of the compound, however the compound still retains its TDF-like functional properties. According to certain embodiments, one or more amino acid residues within the sequence are replaced with other amino acid residues having physical and/or chemical properties similar to the residues they are replacing. Preferably, conservative amino acid substitutions are those wherein an amino acid is replaced with another amino acid encompassed within the same designated class, as will be described more thoroughly below. Insertions, deletions, and substitutions are appropriate where they do not abrogate the functional properties of the compound. Functionality of the altered compound can be assayed according to the in vitro and in vivo assays described below that are designed to assess the TDF-like properties of the altered compound.

According to some embodiments, particularly preferred peptides include but are not limited to, the following:

| Sequence | THR No. | SEQ ID NO: |
|---|---|---|
| CYFDDSSNVLCKKYRS | 123 | 1 |
| CYYDNSSSVLCKRDRS | 204 | 2 |
| CYYDNSSSVLCKRYRS | 184 | 3 |
| CYYDNSSSVLCKKYRS | 194 | 5 |

In yet another embodiment, the peptide of the invention can be:

CYYDNSSSVLCKRX₁₄RS (SEQ ID NO:4), wherein X can be R, H, K, D, E, S, T, N, Q, C, U, G, P, A, V, I, L, M, F, Y, or W, optionally, wherein X₁₄ is D-Tyr.

### TDFRP Recombinant Expression Vectors

According to one aspect, the disclosure includes vectors containing one or more nucleic acid sequences encoding a TDFRP compound. For recombinant expression of one or more the polypeptides of the disclosure, the nucleic acid containing all or a portion of the nucleotide sequence encoding the polypeptide is inserted into an appropriate cloning vector, or an expression vector (i.e., a vector that contains the necessary elements for the transcription and translation of the inserted polypeptide coding sequence) by recombinant DNA techniques well known in the art and as detailed below.

In general, expression vectors useful in recombinant DNA techniques are often in the form of plasmids. In the present specification, “plasmid” and “vector” can be used interchangeably as the plasmid is the most commonly used form of vector. However, the disclosure is intended to include such other forms of expression vectors that are not technically plasmids, such as viral vectors (e.g., replication defective retroviruses, adenoviruses and adeno-associated viruses), which serve equivalent functions. Such viral vectors permit infection of a subject and expression in that subject of a compound.

The recombinant expression vectors of the disclosure comprise a nucleic acid encoding a compound with TDF-like properties in a form suitable for expression of the nucleic acid in a host cell, which means that the recombinant expression vectors include one or more regulatory sequences, selected on the basis of the host cells to be used for expression that is operatively-linked to the nucleic acid sequence to be expressed. Within a recombinant expression vector, “operably-linked” is intended to mean that the nucleotide sequence of interest is linked to the regulatory sequence(s) in a manner that allows for expression of the nucleotide sequence (e.g., in an in vitro transcription/translation system or in a host cell when the vector is introduced into the host cell).

The term “regulatory sequence” is intended to include promoters, enhancers and other expression control elements (e.g., polyadenylation signals). Such regulatory sequences are described, for example, in Goeddel, GENE EXPRESSION TECHNOLOGY: METHODS IN ENZYMOLOGY 185, Academic Press, San Diego, Calif. (1990). Regulatory sequences include those that direct constitutive expression of a nucleotide sequence in many types of host cell and those that direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of polypeptide desired, etc. The expression vectors of the disclosure can be introduced into host cells to thereby produce polypeptides or peptides, including fusion polypeptides, encoded by nucleic acids as described herein (e.g., TDFRP compounds and TDFRP-derived fusion polypeptides, etc.).

### TDFRP-Expressing Host Cells

According to another aspect, the present disclosure pertains to TDFRP-expressing host cells, which contain a nucleic acid encoding one or more TDFRP compounds. The recombinant expression vectors of the disclosure can be designed for expression of TDFRP compounds in prokaryotic or eukaryotic cells. For example. TDFRP compounds can be expressed in bacterial cells such as Escherichia coli, insect cells (using baculovirus expression vectors), fungal cells, e.g., yeast, yeast cells or mammalian cells. Suitable host cells are discussed further in Goeddel, GENE EXPRESSION TECHNOLOGY: METHODS IN ENZYMOLOGY 185, Academic Press, San Diego, CA. (1990). Alternatively, the recombinant expression vector can be transcribed and translated in vitro, for example using T7 promoter regulatory sequences and T7 polymerase.

Expression of polypeptides in prokaryotes is most often carried out in Escherichia coli with vectors containing constitutive or inducible promoters directing the expression of either fusion or non-fusion polypeptides. Fusion vectors add a number of amino acids to a polypeptide encoded therein, usually to the amino terminus of the recombinant polypeptide. Such fusion vectors typically serve three purposes: (i) to increase expression of recombinant polypeptide; (ii) to increase the solubility of the recombinant polypeptide; and (iii) to aid in the purification of the recombinant polypeptide by acting as a ligand in affinity purification. Often, in fusion expression vectors, a proteolytic cleavage site is introduced at the junction of the fusion moiety and the recombinant polypeptide to enable separation of the recombinant polypeptide from the fusion moiety subsequent to purification of the fusion polypeptide. Such enzymes, and their cognate recognition sequences, include Factor Xa, thrombin and enterokinase. Typical fusion expression vectors include pGEX (Pharmacia Biotech Inc; Smith and Johnson, 1988. Gene 67: 31-40), pMAL (New England Biolabs, Beverly, Mass.) and pRIT5 (Pharmacia, Piscataway, N.J.) that fuse glutathione S-transferase (GST), maltose E binding polypeptide, or polypeptide A, respectively, to the target recombinant polypeptide.

Examples of suitable inducible non-fusion E. coli expression vectors include pTre (Amrann et al., (1988) Gene 69:301-315) and pET 11d (Studier et al., GENE EXPRESSION TECHNOLOGY: METHODS IN ENZYMOLOGY 185, Academic Press, San Diego, Calif. (1990) 60-89).

One strategy to maximize recombinant polypeptide expression in E. coli is to express the polypeptide in host bacteria with an impaired capacity to proteolytically cleave the recombinant polypeptide. See, e.g., Gottesman, GENE EXPRESSION TECHNOLOGY: METHODS IN ENZYMOLOGY 185, Academic Press, San Diego, CA. (1990) 119-128. Another strategy is to alter the nucleic acid sequence of the nucleic acid to be inserted into an expression vector so that the individual codons for each amino acid are those preferentially utilized in the expression host, e.g., E. coli (see, e.g., Wada, et al., 1992. Nucl. Acids Res. 20: 2111-2118). Such alteration of nucleic acid sequences of the disclosure can be carried out by standard DNA synthesis techniques.

In another embodiment, the TDFRP expression vector is a yeast expression vector. Examples of vectors for expression in yeast Saccharomyces cerivisae include pYepSec1 (Baldari, et al., 1987. EMBO J. 6: 229-234), pMFa (Kurjan and Herskowitz, 1982. Cell 30: 933-943), pJRY88 (Schultz et al., 1987. Gene 54: 113-123), pYES2 (Invitrogen Corporation, San Diego, CA.), and picZ (Invitrogen Corp, San Diego, CA.). Alternatively, TDFRP can be expressed in insect cells using baculovirus expression vectors. Baculovirus vectors available for expression of polypeptides in cultured insect cells (e.g., SF9 cells) include the pAc series (Smith, et al., 1983. Mol. Cell. Biol. 3: 2156-2165) and the pVL series (Lucklow and Summers, 1989. Virology 170: 31-39).

In yet another embodiment, a nucleic acid of the disclosure is expressed in mammalian cells using a mammalian expression vector. Examples of mammalian expression vectors include pCDM8 (Seed, 1987. Nature 329: 840) and pMT2PC (Kaufman, et al., 1987. EMBO J. 6: 187-195). When used in mammalian cells, the expression vector's control functions are often provided by viral regulatory elements. For example, commonly used promoters are derived from polyoma, adenovirus 2, cytomegalovirus, and simian virus 40. For other suitable expression systems for both prokaryotic and eukaryotic cells see, e.g., Chapters 16 and 17 of Sambrook, et al., MOLECULAR CLONING: A LABORATORY MANUAL. 2^{nd} ed., Cold Spring Harbor Laboratory, Cold Spring Harbor Laboratory Press, Cold Spring Harbor, NY, 1989.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert, et al., 1987. Genes Dev. 1: 268-277), lymphoid-specific promoters (Calame and Eaton, 1988. Adv. Immunol. 43: 235-275), in particular promoters of T cell receptors (Winoto and Baltimore, 1989. EMBO J. 8: 729-733) and immunoglobulins (Banerji, et al., 1983. Cell 33: 729-740; Queen and Baltimore, 1983. Cell 33: 741-748), neuron-specific promoters (e.g., the neurofilament promoter; Byrne and Ruddle, 1989. Proc. Natl. Acad. Sci. USA 86: 5473-5477), pancreas-specific promoters (Edlund, et al., 1985. Science 230: 912-916), and mammary gland-specific promoters (e.g., milk whey promoter; U.S. Pat. No. 4,873,316 and European Application Publication No. 264,166). Developmentally-regulated promoters are also encompassed, e.g., the murine hox promoters (Kessel and Gruss, 1990. Science 249: 374-379) and the α-fetoprotein promoter (Campes and Tilghman, 1989. Genes Dev. 3: 537-546).

The disclosure further provides a recombinant expression vector comprising a DNA molecule of the disclosure cloned into the expression vector in an antisense orientation. That is, the DNA molecule is operatively linked to a regulatory sequence in a manner that allows for expression (by transcription of the DNA molecule) of an RNA molecule that is antisense to a TDRFP mRNA. Regulatory sequences operatively linked to a nucleic acid cloned in the antisense orientation can be chosen that direct the continuous expression of the antisense RNA molecule in a variety of cell types, for instance viral promoters and/or enhancers, or regulatory sequences can be chosen that direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see, e.g., Weintraub, et al., “Antisense RNA as a molecular tool for genetic analysis,” Reviews-Trends in Genetics, Vol. 1(1) 1986.

Another aspect of the disclosure pertains to host cells into which a recombinant expression vector of the disclosure has been introduced. The terms “host cell” and “recombinant host cell” are used interchangeably herein. It is understood that such terms refer not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, TDFRP can be expressed in bacterial cells such as E. coli, insect cells, yeast or mammalian cells (such as Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (MOLECULAR CLONING: A LABORATORY MANUAL. 2^{nd} ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Various selectable markers include those that confer resistance to drugs, such as G418, hygromycin and methotrexate. Nucleic acid encoding a selectable marker can be introduced into a host cell on the same vector as that encoding TDFRP or can be introduced on a separate vector. Cells stably transfected with the introduced nucleic acid can be identified by drug selection (e.g., cells that have incorporated the selectable marker gene will survive, while the other cells die).

A host cell that includes a compound of the disclosure, such as a prokaryotic or eukaryotic host cell in culture can be used to produce (i.e., express) recombinant TDFRP. According to one embodiment, the method comprises culturing the host cell of disclosure (into which a recombinant expression vector encoding TDFRP has been introduced) in a suitable medium such that TDFRP is produced. According to another embodiment, the method further comprises the step of isolating TDFRP from the medium or the host cell. Purification of recombinant polypeptides is well-known in the art and include ion-exchange purification techniques, or affinity purification techniques, for example with an antibody to the compound.

### TDFRP-Derived Chimeric and Fusion Polypeptides

The present disclosure also provides for compounds that are TDFRP-derived chimeric or fusion polypeptides. As used herein, a TDFRP-derived “chimeric polypeptide” or “fusion polypeptide” comprises a TDFRP operatively-linked to a polypeptide having an amino acid sequence corresponding to a polypeptide that is not substantially homologous to the TDFRP, e.g., a polypeptide that is different from the TDFRP and that is derived from the same or a different organism (i.e., non-TDFRP). Within a TDFRP-derived fusion polypeptide, the TDFRP can correspond to all or a portion of a TDFRP. According to one embodiment, a TDFRP-derived fusion polypeptide comprises at least one biologically-active portion of a TDFRP, for example a fragment of SEQ ID NOs:1-347. According to another embodiment, a TDFRP-derived fusion polypeptide comprises at least two biologically active portions of a TDFRP. In yet another embodiment, a TDFRP-derived fusion polypeptide comprises at least three biologically active portions of a TDFRP polypeptide. Within the fusion polypeptide, the term “operatively linked” is intended to indicate that the TDFRP polypeptide and the non-TDFRP polypeptide are fused in-frame with one another. The non-TDFRP polypeptide can be fused to the N-terminus or C-terminus of the TDFRP.

According to one embodiment, the fusion polypeptide is a GST-TDFRP fusion polypeptide in which the TDFRP sequences are fused to the N- or C-terminus of the GST (glutathione S-transferase) sequences. Such fusion polypeptides can facilitate the purification of recombinant TDFRP by affinity means.

According to another embodiment, the fusion polypeptide is a TDFRP polypeptide containing a heterologous signal sequence at its N-terminus. In certain host cells (e.g., mammalian host cells), expression and/or secretion of TDFRP can be increased through use of a heterologous signal sequence.

According to yet another embodiment, the fusion polypeptide is a TDFRP-immunoglobulin fusion polypeptide in which the TDFRP sequences are fused to sequences derived from a member of the immunoglobulin superfamily. The TDFRP-immunoglobulin fusion polypeptides of the disclosure can be incorporated into pharmaceutical compositions and administered to a subject to inhibit an interaction between a TDF and a TDF receptor polypeptide on the surface of a cell, to thereby suppress TDF-mediated signal transduction in vivo. The TDFRP-immunoglobulin fusion polypeptides can be used to affect the bioavailability of a TDFRP, for example to target the compound to a particular cell or tissue having the requisite antigen. Inhibition of the TDF/TDF receptor interaction can be useful therapeutically for both the treatment of proliferative and differentiative disorders, as well as modulating (e.g., promoting or inhibiting) cell survival.

### TDFRP1-Linker-TDFRP2

The TDFRP compounds described herein contain multiple TDF-related polypeptides (i.e., multiple domain TDF-related polypeptide compounds, hereinafter “TDFRP”) with the general structure shown below:
TDFRP1-linker-TDFRP2
   Where a first TDFRP domain (TDFRP1, i.e., TDF-related polypeptide 1) is covalently linked via the C-terminus, N-terminus, or any position with a functionalizable side group, e.g., lysine or aspartic acid to a linker molecule, which, in turn, is covalently linked to the N-terminus of a second TDFRP domain (TDFRP2).

The TDRFP domains are compounds that include small molecules. Variants, analogs, homologs, or fragments of these compounds, such as species homologs, are also included in the present disclosure, as well as degenerate forms thereof.

A first domain is linked to a second domain through a linker. The term “linker,” as used herein, refers to an element capable of providing appropriate spacing or structural rigidity, or structural orientation, alone, or in combination, to a first and a second domain, e.g., TDFRP1 and TDFRP2, such that the biological activity of the TDFRP is preserved. For example, linkers may include, but are not limited to, a diamino alkane, a dicarboxylic acid, an amino carboxylic acid alkane, an amino acid sequence, e.g., glycine polypeptide, a disulfide linkage, a helical or sheet-like structural element or an alkyl chain. According to one aspect the linker is not inert, e.g., chemically or enzymatically cleavable in vivo or in vitro. In another aspect the linker is inert, i.e., substantially unreactive in vivo or in vitro, e.g., is not chemically or enzymatically degraded. Examples of inert groups which can serve as linking groups include aliphatic chains such as alkyl, alkenyl and alkynyl groups (e.g., C1-C20), cycloalkyl rings (e.g., C3-C10), aryl groups (carbocyclic aryl groups such as 1-naphthyl, 2-naphthyl, 1-anthracyl and 2-anthracyl and heteroaryl group such as N-imidazolyl, 2-imidazole, 2-thienyl, 3-thienyl, 2-furanyl, 3-furanyl, 2-pyridyl, 3-pyridyl, 4-pyridyl, 2-pyrimidy, 4-pyrimidyl, 2-pyranyl, 3-pyranyl, 3-pyrazolyl, 4-pyrazolyl, 5-pyrazolyl, 2-pyrazinyl, 2-thiazole, 4-thiazole, 5-thiazole, 2-oxazolyl, 4-oxazolyl, 5-oxazolyl, 2-benzothienyl, 3-benzothienyl, 2-benzofuranyl, 3-benzofuranyl, 2-indolyl, 3-indolyl, 2-quinolinyl, 3-quinolinyl, 2-benzothiazole, 2-benzooxazole, 2-benzimidazole, 2-quinolinyl, 3-quinolinyl, 1-isoquinolinyl, 3-quinolinyl, 1-isoindolyl, and 3-isoindolyl), non-aromatic heterocyclic groups (e.g., 2-tetrahydrofuranyl, 3-tetrahydrofuranyl, 2-tetrahyrothiophenyl, 3-tetrahyrothiophenyl, 2-morpholino, 3-morpholino, 4-morpholino, 2-thiomorpholino, 3-thiomorpholino, 4-thiomorpholino, 1-pyrrolidinyl, 2-pyrrolidinyl, 3-pyrrolidinyl, 1-piperazinyl, 2-piperazinyl, 1-piperidinyl, 2-piperidinyl, 3-piperidinyl, 4-piperidinyl and 4-thiazolidinyl) and aliphatic groups in which one, two or three methylenes have been replaced with —O—, —S—, —NH—, —SO₂—, —SO— or —SO₂NH—.

The TDFRP compounds include small molecules, more particularly TDFRP compound domains, with the general structure identified herein, as detailed below. The TDFRP compound domains disclosed herein may be present in an TDFRP compound in any combination or orientation. Variants, analogs, homologs, or fragments of these TDFRP compound domains, such as species homologs, are also included in the present disclosure, as well as degenerate forms thereof. The TDFRP compound domains of the present disclosure may be capped on the N-terminus, or the C-terminus, or on both the N-terminus and the C-terminus. The TDFRP compounds may be pegylated, or modified, e.g., branching, at any amino acid residue containing a reactive side chain, e.g., lysine residue, or chemically reactive group on the linker. The TDFRP compound of the present disclosure may be linear or cyclized. The tail sequence of the TDFRP or TDFRP domains may vary in length. According to one aspect of the present disclosure, the TDFRP compounds of the disclosure are prodrugs, i.e., the biological activity of the TDFRP compound is altered, e.g., increased, upon contacting a biological system in vivo or in vitro.

The TDFRP compounds can contain natural amino acids, non-natural amino acids, d-amino acids and l-amino acids, and any combinations thereof. According to certain embodiments, the compounds of the disclosure can include commonly encountered amino acids, which are not genetically encoded. These non-genetically encoded amino acids include, but are not limited to, β-alanine (β-Ala) and other omega-amino acids such as 3-aminopropionic acid (Dap), 2,3-diaminopropionic acid (Dpr), 4-aminobutyric acid and so forth; α-aminoisobutyric acid (Aib); ε-aminohexanoic acid (Aha); δ-aminovaleric acid (Ava); N-methylglycine or sarcosine (MeGly); ornithine (Orn); citrulline (Cit); t-butylalanine (t-BuA); t-butylglycine (t-BuG); N-methylisoleucine (MeIle); phenylglycine (Phg); cyclohexylalanine (Cha); norleucine (Nle); 2-naphthylalanine (2-Nal); 4-chlorophenylalanine (Phe(4-Cl)); 2-fluorophenylalanine (Phe(2-F)); 3-fluorophenylalanine (Phe(3-F)); 4-fluorophenylalanine (Phe(4-F)); penicillamine (Pen); 1,2,3,4-tetrahydroisoquinoline-3-carboxylic acid (Tic); β-2-thienylalanine (Thi); methionine sulfoxide (MSO); homoarginine (hArg); N-acetyl lysine (AcLys); 2,3-diaminobutyric acid (Dab); 2,3-diaminobutyric acid (Dbu); p-aminophenylalanine (Phe(pNH₂)); N-methyl valine (MeVal); homocysteine (hCys) and homoserine (hSer). Non-naturally occurring variants of the compounds may be produced by mutagenesis techniques or by direct synthesis.

### Measurement of TDFRP Biological Activity

The biological activity, namely the agonist or antagonist properties of TDF polypeptides or TDFRP compounds can be characterized using any conventional in vivo and in vitro assays that have been developed to measure the biological activity of the TDFRP compound, a TDF polypeptide or a TDF signaling pathway component.

TGF-b/BMPs Superfamily members are associated with a number of cellular activities involved in injury responses and regeneration. TDFRP compounds can be used as agonists of BMPs or antagonists of TGF-b molecules to mediate activities that can prevent, repair or alleviate injurious responses in cells, tissues or organs. According to some embodiments, the anti-cancer properties of the TDFRPs is measured. According to some embodiments, the anti-cancer properties of TDFRP compounds can been demonstrated using in vitro and in vivo assays, e.g., blood test, biopsy, X-ray, CT scan.

### Methods

The present disclosure provides TDFRPs compounds that are functional analogs of tissue differentiation factors, i.e., compounds that functionally mimic TGF-beta superfamily proteins, for example by acting as TGF-beta superfamily receptor agonists.

The disclosure provides for both prophylactic and therapeutic methods of treating a subject at risk of (or susceptible to) a disorder or having a disorder associated with aberrant TDF polypeptide or TDFRP compound target molecule expression or activity. TDF and TDFRP compound target molecules, such as TDF receptors, play a role in cell differentiation. Cell differentiation is the central characteristic of tissue morphogenesis. Tissue morphogenesis is a process involved in adult tissue repair and regeneration mechanisms. The degree of morphogenesis in adult tissue varies among different tissues and is related, among other things, to the degree of cell turnover in a given tissue.

The bone morphogenetic proteins are members of the transforming growth factor-beta superfamily. Ozkaynak et al. (EMBO J. 9: 2085-2093, 1990) purified a novel bovine osteogenic protein homolog, which they termed ‘osteogenic protein-1’ (OP-1; a.k.a., BMP-7). The authors used peptide sequences to clone the human genomic and cDNA clones of OP-1, later named BMP-7. The BMP-7 cDNAs predicted a 431-amino acid polypeptide that includes a secretory signal sequence. The TDFRP compounds described herein are structural mimetics of the biologically active regions of bone morphogenetic proteins, for example, but not limited to, BMP-7 (OP-1), and related peptides. Biologically active regions include, for example, the Finger 1 and Finger 2 regions of BMP-7. Groppe et al. (Nature 420: 636-642, 2002) reported the crystal structure of the antagonist Noggin (602991) bound to BMP-7.

TDFRP compounds are useful to treat diseases and disorders that are amenable to treatment with BMP polypeptides. According to some embodiments, the TDFRP compounds of the disclosure are useful to alter, e.g., inhibit or accelerate, the ability to repair and regenerate diseased or damaged tissues and organs, as well as, to treat TDF-associated disorders. Particularly useful areas for TDFRP-based human and veterinary therapeutics include reconstructive surgery, the treatment of tissue degenerative diseases including, for example, renal disease, brain trauma, stroke, atherosclerosis, arthritis, emphysema, osteoporosis, cardiomyopathy, cirrhosis, degenerative nerve diseases, inflammatory diseases, and cancer, and in the regeneration of tissues, organs and limbs. The TDFRP compounds of the disclosure can also be used to promote or inhibit the growth and differentiation of muscle, bone, skin, epithelial, heart, nerve, endocrine, vessel, cartilage, periodontal, liver, retinal, and connective tissue, or any tissue where functional TDRFP compound target molecules are expressed. Accordingly, diseases associated with aberrant TDF polypeptide or TDFRP compound target molecule expression include viral infections, cancer, healing, neurodegenerative disorders, e.g., Alzheimer's Disease, Parkinson's Disorder, immune disorders, and bone disorders. For example, TDFRP-based therapeutic compositions are useful to induce regenerative healing of bone defects such as fractures, as well as, to preserve or restoring healthy metabolic properties in diseased tissue, e.g., osteopenic bone tissue.

TDFRP compounds are useful to treat diseases and disorders that are amenable to treatment with BMP polypeptides. According to some embodiments, the TDFRP compounds of the disclosure are useful to treat pulmonary cancer.

### Diseases and Disorders

According to some embodiments, the methods and compositions disclosed herein can treat cancer. In some embodiments, the cancer is breast cancer, prostate cancer, renal cell carcinoma, bone metastasis, lung cancer or metastasis, osteosarcoma, multiple myeloma, astrocytoma, pilocytic astrocytoma, dysembryoplastic neuroepithelial tumor, oligodendrogliomas, ependymoma, glioblastoma multiforme, mixed gliomas, oligoastrocytomas, medulloblastoma, retinoblastoma, neuroblastoma, germinoma, teratoma, gangliogliomas, gangliocytoma, central gangliocytoma, primitive neuroectodermal tumors (PNET, e.g. medulloblastoma, medulloepithelioma, neuroblastoma, retinoblastoma, ependymoblastoma), tumors of the pineal parenchyma (e.g. pineocytoma, pineoblastoma), ependymal cell tumors, choroid plexus tumors, neuroepithelial tumors of uncertain origin (e.g. gliomatosis cerebri, astroblastoma), esophageal cancer, colorectal cancer, CNS, ovarian, melanoma pancreatic cancer, squamous cell carcinoma, hematologic cancer (e.g., leukemia, lymphoma, and multiple myeloma), colon cancer, rectum cancer, stomach cancer, kidney cancer, mesothelioma, bladder cancer, skin cancer, and/or a combination thereof. In some embodiments, the cancer is breast cancer. In some embodiments, the cancer is prostate cancer.

According to some embodiments, the disclosure provides for methods to treat a subject having cancer with mature dendritic cells, with or without one or more TDFRPs. In some embodiments, the mature dendritic cells are produced by providing monocytes from the subject having cancer and contacting the monocytes with one or more TDFRPs, thereby producing immature dendritic cells. In some embodiments, the monocytes are contacted with interleukin 4 (IL-4) and Granulocyte-macrophage colony-stimulating factor (GM-CSF) and one or more TDFRPs, thereby producing immature dendritic cells. In some embodiments, the immature dendritic cells are contacted with one or more TDFRPs, thereby producing mature dendritic cells. In some embodiments, the immature dendritic cells are contacted with one or more TDFRPs and lipopolysaccharide, thereby producing mature dendritic cells.

According to some embodiments, the one or more TDFRPs comprise amino acid sequence SEQ ID NO: 1 or SEQ ID NO: 3. In some embodiments, the one or more TDFRPs comprise amino acid sequence SEQ ID NO: 1. In some embodiments, the one or more TDFRPs comprise amino acid sequence SEQ ID NO: 3. In some embodiments, the one or more TDFRPs comprise amino acid sequence SEQ ID NO: 1 and SEQ ID NO: 3. In some embodiments, the one or more TDFRPs comprise a crosslink.

According to some embodiments, the disclosure provides for methods to treat a subject having cancer with mature dendritic cells and one or more TDFRPs, thereby rendering a cancer cell responsive to one or more antineoplastic modalities. In some embodiments, the mature dendritic cells are produced by providing monocytes from the subject having cancer and contacting the monocytes with one or more TDFRPs, thereby producing immature dendritic cells. In some embodiments, the monocytes are contacted with interleukin 4 (IL-4) and Granulocyte-macrophage colony-stimulating factor (GM-CSF) and one or more TDFRPs, thereby producing immature dendritic cells. In some embodiments, the immature dendritic cells are contacted with one or more TDFRPs, thereby producing mature dendritic cells. In some embodiments, the immature dendritic cells are contacted with one or more TDFRPs and lipopolysaccharide, thereby producing mature dendritic cells.

According to some embodiments, the one or more antineoplastic modalities are one or more chemotherapeutic agents, surgery, radiation, immunotherapy, hormone therapy, stem cell transplant, small-molecules, antibodies, chimeric antigen receptor T cells (CAR-T cells), cancer vaccines, or a combination thereof.

According to some embodiments, the disclosure provides for methods of increasing T-cell recognition of tumor cell antigens on tumor cells in a subject having cancer by administering to the subject an effective amount of the isolated population of mature dendritic cells of the invention, thereby decreasing TGF-beta secretion from the tumor cells compared to a control tumor cell. In some embodiments, the one or more TDFRPs are administered to the subject with the mature dendritic cells of the invention.

According to some embodiments of the disclosures and aspects herein, the TDFRP is selected from the group consisting of: SEQ ID NO: 1, SEQ ID NO: 2 and SEQ ID NO: 3.

In some embodiments, the TDFRP comprises the peptide having the sequence of SEQ ID NO: 1. In some embodiments, the TDFRP comprises an amino acid sequence at least 85%, 90%, 95%, 96%, 97%, 98% or 99% identical to SEQ ID NO: 1. In some embodiments, the TDFPR comprises an amino acid sequence consisting of SEQ ID NO: 1.

In some embodiments, the TDFRP comprises the peptide having the sequence of SEQ ID NO: 2. In some embodiments, the TDFRP comprises an amino acid sequence at least 85%, 90%, 95%, 96%, 97%, 98% or 99% identical to SEQ ID NO: 2. In some embodiments, the TDFPR comprises an amino acid sequence consisting of SEQ ID NO: 2.

In some embodiments, the TDFRP comprises the peptide having the sequence of SEQ ID NO: 3. In some embodiments, the TDFRP comprises an amino acid sequence at least 85%, 90%, 95%, 96%, 97%, 98% or 99% identical to SEQ ID NO: 3. In some embodiments, the TDFPR comprises an amino acid sequence consisting of SEQ ID NO: 3.

In some embodiments, the TDFRP comprises the peptide having the sequence of SEQ ID NO: 4. In some embodiments, the TDFRP comprises an amino acid sequence at least 85%, 90%, 95%, 96%, 97%, 98% or 99% identical to SEQ ID NO: 4. In some embodiments, the TDFPR comprises an amino acid sequence consisting of SEQ ID NO: 4.

In some embodiments, the TDFRP comprises the peptide having the sequence of SEQ ID NO: 5. In some embodiments, the TDFRP comprises an amino acid sequence at least 85%, 90%, 95%, 96%, 97%, 98% or 99% identical to SEQ ID NO: 5. In some embodiments, the TDFPR comprises an amino acid sequence consisting of SEQ ID NO: 5.

According to some embodiments of the embodiments and aspects herein, the TDFRP is a Multiple Domain TDFRP. In some embodiments, the TDFRP comprises a crosslink. According to some embodiments, the TDFPR is administered with an additional agent. According to one embodiment, the serum half-life of the TDFRP is increased when administered with the additional agent, compared to administration of the TDFRP alone.

### Mature Dendritic Cells

According to some embodiments of the disclosures and aspects herein, methods are provided to produce mature dendritic cells, the method comprising a) providing monocytes from a subject having cancer; b) contacting the monocytes with one or more tissue differentiation factor related polypeptides (TDFRPs), thereby producing immature dendritic cells; c) contacting the immature dendritic cells with one or more TDFRPs, thereby producing mature dendritic cells; and d) isolating mature dendritic cells.

According to some embodiments, step b) further comprises contacting the monocytes with interleukin 4 (IL-4) and Granulocyte-macrophage colony-stimulating factor (GM-CSF). Monocytes may be differentiated into dendritic cells (DCs) by any technique well known in the art. For example granulocyte-macrophage colony-stimulating factor (GM-CSF) with interleukin-4 (IL-4) differentiates monocytes into DCs. Multiple methods well known to the art have been described to differentiate human blood monocytes into dendritic cells by using GM-CSF, IL-4, and/or IFN-γ and/or CD40 ligation (Gieseler R et al. 1998, Zhou et al. 1996; Cahpuis et al 1997, Brossart et al. 1998, Palucka et al 1998). LC cells, a DC subset may be derived using TGF-β in addition (Strobl et al. 1997).

According to some embodiments, step c) further comprises contacting the immature dendritic cells with lipopolysaccharide. Lipopolysaccharide (LPS) or endotoxin is a predominant integral structural component of the outer membrane of Gram-negative bacteria and one of the most potent microbial initiators of inflammation. LPS binds to the CD14 glycoprotein that is expressed on the surface of monocytes and stimulates the toll receptor pathway via activation of TLR4. Other PAMPs (pathogen associated molecular patterns) can also initiate inflammatory responses via other TLR receptors. The binding of LPS or other PAMPS induces production of TNF-α, IL-1, -6, -8 and -10 (Wright S D. Et al; 1990; Dobrovolskaia M A et al. 2002; Foey A D. et al. 2000).

According to some embodiments, the monocytes of step b) are cultured for at least 2 days, at least 3 days, at least 4 days at least 5 days, at least 6 days, or at least 7 days. According to some embodiments, the monocytes of step b) are cultured for about 2 says, about 3 days, about 4 days, about 5 days, about 6 days, about 7 days, or about 8 days.

According to some embodiments, the immature dendritic cells of step c) are cultured for about 24 hours, about 36 hours, about 48 hours, about 60 hours, or about 72 hours. According to some embodiments, the immature dendritic cells of step c) are cultured for at least 24 hours, at least 36 hours, at least 48 hours, at least 60 hours, or at least 72 hours.

According to some embodiments, the one or more TDFRPs comprises amino acid sequence SEQ ID NO: 1 or 3.

In some embodiments, the TDFRP comprises the peptide having the sequence of SEQ ID NO: 1. In some embodiments, the TDFRP comprises an amino acid sequence at least 85%, 90%, 95%, 96%, 97%, 98% or 99% identical to SEQ ID NO: 1. In some embodiments, the TDFPR comprises an amino acid sequence consisting of SEQ ID NO: 1.

In some embodiments, the TDFRP comprises the peptide having the sequence of SEQ ID NO: 2. In some embodiments, the TDFRP comprises an amino acid sequence at least 85%, 90%, 95%, 96%, 97%, 98% or 99% identical to SEQ ID NO: 2. In some embodiments, the TDFPR comprises an amino acid sequence consisting of SEQ ID NO: 2.

In some embodiments, the TDFRP comprises the peptide having the sequence of SEQ ID NO: 3. In some embodiments, the TDFRP comprises an amino acid sequence at least 85%, 90%, 95%, 96%, 97%, 98% or 99% identical to SEQ ID NO: 3. In some embodiments, the TDFPR comprises an amino acid sequence consisting of SEQ ID NO: 3.

In some embodiments, the TDFRP comprises the peptide having the sequence of SEQ ID NO: 4. In some embodiments, the TDFRP comprises an amino acid sequence at least 85%, 90%, 95%, 96%, 97%, 98% or 99% identical to SEQ ID NO: 4. In some embodiments, the TDFPR comprises an amino acid sequence consisting of SEQ ID NO: 4.

In some embodiments, the TDFRP comprises the peptide having the sequence of SEQ ID NO: 5. In some embodiments, the TDFRP comprises an amino acid sequence at least 85%, 90%, 95%, 96%, 97%, 98% or 99% identical to SEQ ID NO: 5. In some embodiments, the TDFPR comprises an amino acid sequence consisting of SEQ ID NO: 5.

According to some embodiments, the one or more TDFRPs comprise a crosslink.

According to some embodiments, the cancer comprises cancer cells expressing BMP receptors. According to some embodiments, the cancer is breast cancer, prostate cancer, renal cell carcinoma, bone metastasis, lung cancer or metastasis, osteosarcoma, multiple myeloma, astrocytoma, pilocytic astrocytoma, dysembryoplastic neuroepithelial tumor, oligodendrogliomas, ependymoma, glioblastoma multiforme, mixed gliomas, oligoastrocytomas, medulloblastoma, retinoblastoma, neuroblastoma, germinoma, teratoma, gangliogliomas, gangliocytoma, central gangliocytoma, primitive neuroectodermal tumors (PNET, e.g. medulloblastoma, medulloepithelioma, neuroblastoma, retinoblastoma, ependymoblastoma), tumors of the pineal parenchyma (e.g. pineocytoma, pineoblastoma), ependymal cell tumors, choroid plexus tumors, neuroepithelial tumors of uncertain origin (e.g. gliomatosis cerebri, astroblastoma), esophageal cancer, colorectal cancer, CNS, ovarian, melanoma pancreatic cancer, squamous cell carcinoma, hematologic cancer (e.g., leukemia, lymphoma, and multiple myeloma), colon cancer, rectum cancer, stomach cancer, kidney cancer, mesothelioma, bladder cancer, skin cancer, or a combination thereof. According to come embodiments, the cancer is breast cancer. According to some embodiments, the cancer is prostate cancer.

In some embodiments, the cancer is a solid tumor or a hematologic cancer. In some embodiments, the cancer is a solid tumor. In some embodiments, the cancer is a hematologic cancer.

According to some embodiments of the disclosures and aspects herein, a pharmaceutical composition is provided comprising the mature dendritic cells and/or one or more TDFRPs as described herein. In some embodiments, the pharmaceutical composition is administered to a subject having cancer to treat the cancer. In some embodiments, the mature dendritic cells of the pharmaceutical composition was produced from monocytes collected from the subject. In some embodiments, the monocytes are collected for a donor.

According to some embodiments, the one or more TDFRPs comprises amino acid sequence SEQ ID NO: 1 or 3.

In some embodiments, the TDFRP comprises the peptide having the sequence of SEQ ID NO: 1. In some embodiments, the TDFRP comprises an amino acid sequence at least 85%, 90%, 95%, 96%, 97%, 98% or 99% identical to SEQ ID NO: 1. In some embodiments, the TDFPR comprises an amino acid sequence consisting of SEQ ID NO: 1.

In some embodiments, the TDFRP comprises the peptide having the sequence of SEQ ID NO: 2. In some embodiments, the TDFRP comprises an amino acid sequence at least 85%, 90%, 95%, 96%, 97%, 98% or 99% identical to SEQ ID NO: 2. In some embodiments, the TDFPR comprises an amino acid sequence consisting of SEQ ID NO: 2.

In some embodiments, the TDFRP comprises the peptide having the sequence of SEQ ID NO: 3. In some embodiments, the TDFRP comprises an amino acid sequence at least 85%, 90%, 95%, 96%, 97%, 98% or 99% identical to SEQ ID NO: 3. In some embodiments, the TDFPR comprises an amino acid sequence consisting of SEQ ID NO: 3.

In some embodiments, the TDFRP comprises the peptide having the sequence of SEQ ID NO: 4. In some embodiments, the TDFRP comprises an amino acid sequence at least 85%, 90%, 95%, 96%, 97%, 98% or 99% identical to SEQ ID NO: 4. In some embodiments, the TDFPR comprises an amino acid sequence consisting of SEQ ID NO: 4.

In some embodiments, the TDFRP comprises the peptide having the sequence of SEQ ID NO: 5. In some embodiments, the TDFRP comprises an amino acid sequence at least 85%, 90%, 95%, 96%, 97%, 98% or 99% identical to SEQ ID NO: 5. In some embodiments, the TDFPR comprises an amino acid sequence consisting of SEQ ID NO: 5.

According to some embodiments, the one or more TDFRPs comprise a crosslink.

In some embodiments, the cancer is breast cancer, prostate cancer, renal cell carcinoma, bone metastasis, lung cancer or metastasis, osteosarcoma, multiple myeloma, astrocytoma, pilocytic astrocytoma, dysembryoplastic neuroepithelial tumor, oligodendrogliomas, ependymoma, glioblastoma multiforme, mixed gliomas, oligoastrocytomas, medulloblastoma, retinoblastoma, neuroblastoma, germinoma, teratoma, gangliogliomas, gangliocytoma, central gangliocytoma, primitive neuroectodermal tumors (PNET, e.g. medulloblastoma, medulloepithelioma, neuroblastoma, retinoblastoma, ependymoblastoma), tumors of the pineal parenchyma (e.g. pincocytoma, pineoblastoma), ependymal cell tumors, choroid plexus tumors, neuroepithelial tumors of uncertain origin (e.g. gliomatosis cerebri, astroblastoma), esophageal cancer, colorectal cancer, CNS, ovarian, melanoma pancreatic cancer, squamous cell carcinoma, hematologic cancer (e.g., leukemia, lymphoma, and multiple myeloma), colon cancer, rectum cancer, stomach cancer, kidney cancer, mesothelioma, bladder cancer, skin cancer, or a combination thereof. In some embodiments, the cancer is a solid tumor. In some embodiments, the cancer is hematologic cancer.

According to some embodiments of the disclosures and aspects herein, a method of treating a subject having cancer is provided comprising administering a pharmaceutical composition of the mature dendritic cells and/or one or more TDFRPs as described herein.

In some embodiments, the pharmaceutical composition is administered to a subject having cancer to treat the cancer. In some embodiments, the mature dendritic cells of the pharmaceutical composition was produced from monocytes collected from the subject. In some embodiments, the monocytes are collected for a donor.

According to some embodiments, the one or more TDFRPs comprises amino acid sequence SEQ ID NO: 1 or 3.

In some embodiments, the TDFRP comprises the peptide having the sequence of SEQ ID NO: 1. In some embodiments, the TDFRP comprises an amino acid sequence at least 85%, 90%, 95%, 96%, 97%, 98% or 99% identical to SEQ ID NO: 1. In some embodiments, the TDFPR comprises an amino acid sequence consisting of SEQ ID NO: 1.

In some embodiments, the TDFRP comprises the peptide having the sequence of SEQ ID NO: 2. In some embodiments, the TDFRP comprises an amino acid sequence at least 85%, 90%, 95%, 96%, 97%, 98% or 99% identical to SEQ ID NO: 2. In some embodiments, the TDFPR comprises an amino acid sequence consisting of SEQ ID NO: 2.

In some embodiments, the TDFRP comprises the peptide having the sequence of SEQ ID NO: 3. In some embodiments, the TDFRP comprises an amino acid sequence at least 85%, 90%, 95%, 96%, 97%, 98% or 99% identical to SEQ ID NO: 3. In some embodiments, the TDFPR comprises an amino acid sequence consisting of SEQ ID NO: 3.

In some embodiments, the TDFRP comprises the peptide having the sequence of SEQ ID NO: 4. In some embodiments, the TDFRP comprises an amino acid sequence at least 85%, 90%, 95%, 96%, 97%, 98% or 99% identical to SEQ ID NO: 4. In some embodiments, the TDFPR comprises an amino acid sequence consisting of SEQ ID NO: 4.

In some embodiments, the TDFRP comprises the peptide having the sequence of SEQ ID NO: 5. In some embodiments, the TDFRP comprises an amino acid sequence at least 85%, 90%, 95%, 96%, 97%, 98% or 99% identical to SEQ ID NO: 5. In some embodiments, the TDFPR comprises an amino acid sequence consisting of SEQ ID NO: 5.

According to some embodiments, the one or more TDFRPs comprise a crosslink.

In some embodiments, the method reduces the severity of one or more symptoms associated with the cancer by at least about any of 10%, 20%, 30%, 40%, 50%, 60%, 70%, 80%, 90%, 95% or 100% compared to the corresponding symptom in the same individual prior to treatment or compared to the corresponding symptom in other individuals not receiving the treatment method. In some embodiments, the method delays progression of the cancer.

According to some embodiments of the disclosures and aspects herein, a method of treating a subject having cancer comprising administering one or more tissue differentiation factor related polypeptides (TDFRPs) to the subject, thereby rendering a cancer cell responsive to one or more antineoplastic modalities.

In some embodiments, the pharmaceutical composition is administered to a subject having cancer to treat the cancer. In some embodiments, the mature dendritic cells of the pharmaceutical composition was produced from monocytes collected from the subject. In some embodiments, the monocytes are collected for a donor.

In some embodiments, the cancer is breast cancer, prostate cancer, renal cell carcinoma, bone metastasis, lung cancer or metastasis, osteosarcoma, multiple myeloma, astrocytoma, pilocytic astrocytoma, dysembryoplastic neuroepithelial tumor, oligodendrogliomas, ependymoma, glioblastoma multiforme, mixed gliomas, oligoastrocytomas, medulloblastoma, retinoblastoma, neuroblastoma, germinoma, teratoma, gangliogliomas, gangliocytoma, central gangliocytoma, primitive neuroectodermal tumors (PNET, e.g. medulloblastoma, medulloepithelioma, neuroblastoma, retinoblastoma, ependymoblastoma), tumors of the pineal parenchyma (e.g. pineocytoma, pineoblastoma), ependymal cell tumors, choroid plexus tumors, neuroepithelial tumors of uncertain origin (e.g. gliomatosis cerebri, astroblastoma), esophageal cancer, colorectal cancer, CNS, ovarian, melanoma pancreatic cancer, squamous cell carcinoma, hematologic cancer (e.g., leukemia, lymphoma, and multiple myeloma), colon cancer, rectum cancer, stomach cancer, kidney cancer, mesothelioma, bladder cancer, skin cancer, or a combination thereof. In some embodiments, the cancer is a solid tumor. In some embodiments, the cancer is hematologic cancer.

In some embodiments, the cancer cells are resistant to one or more antineoplastic modalities. In some embodiments, the one or more antineoplastic modalities are one or more chemotherapeutic agents, surgery, radiation, immunotherapy, hormone therapy, stem cell transplant, small-molecules, antibodies, chimeric antigen receptor T cells (CAR-T cells), cancer vaccines, or a combination thereof.

In some embodiments, the responsiveness of the cancer cells to one or more antineoplastic modalities is determined by, but not limited to, blood test, X-ray, and CT scan. In some embodiments, the method reduces the severity of one or more symptoms associated with the cancer by at least about any of 10%, 20%, 30%, 40%, 50%, 60%, 70%, 80%, 90%, 95% or 100% compared to the corresponding symptom in the same individual prior to treatment or compared to the corresponding symptom in other individuals not receiving the treatment method. In some embodiments, the method delays progression of the cancer.

According to some embodiments of the disclosures and aspects herein, an in vivo method of increasing T-cell recognition of tumor cell antigens on tumor cells in a subject having cancer, comprising administering to the subject an effective amount of the isolated population of mature dendritic cells as described herein, wherein TGF-beta secretion from the tumor cells is decreased compared to a control tumor cell.

In some embodiments, the TGF-beta secretion is decreased by about 1.2-fold, about 2.5-fold, about 2-fold, about 2.2-fold, about 2.5-fold, about 3-fold, about 3.2-fold, about 3.5-fold, about 4-fold, about 4.2-fold, about 5-fold, about 6-fold, about 7-fold, about 8-fold, about 9-fold, or about 10-fold in comparison to a control tumor cell.

### Biological Effect

In various embodiments of the disclosure, suitable in vitro or in vivo assays are performed to determine the effect of a specific TDFRP-based therapeutic, and whether its administration is indicated for treatment of the affected tissue in a subject.

In various specific embodiments, in vitro assays can be performed with representative cells of the type(s) involved in the patient's disorder, to determine if a given TDFRP-based therapeutic exerts the desired effect upon the cell type(s). Compounds for use in therapy can be tested in suitable animal model systems including, but not limited to rats, mice, chicken, cows, monkeys, rabbits, and the like, prior to testing in human subjects. Similarly, for in vivo testing, any of the animal model system known in the art can be used prior to administration to human subjects.

### Pharmaceutical Compositions

The pharmaceutical compositions of the disclosure typically contain a therapeutically effective amount of a compound described herein. Those skilled in the art will recognize, however, that a pharmaceutical composition may contain more than a therapeutically effective amount, such as in bulk compositions, or less than a therapeutically effective amount, that is, individual unit doses designed for multiple administration to achieve a therapeutically effective amount. Typically, the composition will contain from about 0.01-95 wt % of active agent, including, from about 0.01-30 wt %, such as from about 0.01-10 wt %, with the actual amount depending upon the formulation itself, the route of administration, the frequency of dosing, and so forth. According to one embodiment, a composition suitable for an oral dosage form, for example, may contain about 5-70 wt %, or from about 10-60 wt % of active agent.

According to some aspects, the TDFRP compounds of the disclosure, and derivatives, fragments, analogs and homologs thereof, are be incorporated into pharmaceutical compositions suitable for administration.

According to some embodiments, the pharmaceutical composition includes one or more TDFPR compounds and an additional agent. According to some embodiments, the TDFRP compositions of the disclosure may be physically mixed with the additional agent to form a composition containing both agents; or the TDFRP and additional agent each may be present in separate and distinct compositions which are administered to the patient simultaneously or at separate times. For example, a TDFRP compound of the disclosure can be combined with an additional agent using conventional procedures and equipment to form a combination of active agents comprising a TDFRP compound of the disclosure and an additional agent. Additionally, the additional agents may be combined with a pharmaceutically acceptable carrier to form a pharmaceutical composition comprising a compound of the disclosure, a second active agent and a pharmaceutically acceptable carrier. In this embodiment, the components of the composition are typically mixed or blended to create a physical mixture. The physical mixture is then administered in a therapeutically effective amount using any of the routes described herein.

Alternatively, the TDFRP compound and additional agent may remain separate and distinct before administration to the patient. In this embodiment, the TDFRP compound and additional agent are not physically mixed together before administration but are administered simultaneously or at separate times as separate compositions. Such compositions can be packaged separately or may be packaged together in a kit. When administered at separate times, the additional agent will typically be administered less than 24 hours after administration of the compound of the disclosure, ranging anywhere from concurrent with administration of the compound of the disclosure to about 24 hours post-dose. This is also referred to as sequential administration. Thus, a compound of the disclosure can be orally administered simultaneously or sequentially with the additional agent using two tablets, with one tablet for the TDFRP compound and one tablet for the additional agent, where sequential may mean being administered immediately after administration of the compound of the disclosure or at some predetermined time later (for example, one hour later or three hours later). It is also contemplated that the additional agent may be administered more than 24 hours after administration of the compound of the disclosure. Alternatively, the combination may be administered by different routes of administration, that is, one orally and the other by inhalation.

According to one embodiment, the TDFRP is provided in the same pharmaceutical composition as the additional agent. According to another embodiment, the TDFRP is provided in a separate composition as the additional agent.

As used herein, “pharmaceutically acceptable carrier” is intended to include any and all solvents, dispersion media, coatings, antibacterial and antifungal compounds, isotonic and absorption delaying compounds, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, Ringer's solutions, dextrose solution, and 5% human serum albumin. Liposomes and non-aqueous vehicles such as fixed oils may also be used. The use of such media and compounds for pharmaceutically active substances is well known in the art. Except insofar as any conventional media or compound is incompatible with the active compound, use thereof in the compositions is contemplated. Supplementary active compounds can also be incorporated into the compositions.

A pharmaceutical composition of the disclosure is formulated to be compatible with its intended route of administration. Examples of routes of administration include parenteral, e.g., intravenous, intradermal, subcutaneous, oral (e.g., inhalation), transdermal (i.e., topical), transmucosal, and rectal administration. According to some embodiments, a pharmaceutical composition is formulated to be compatible with intravenous, intraperitoneal, intramuscular, subcutaneous, inhalation, transmucosal, and oral routes of administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial compounds such as benzyl alcohol or methyl parabens; antioxidants such as ascorbic acid or sodium bisulfite; chelating compounds such as ethylenediaminetetraacetic acid (EDTA); buffers such as acetates, citrates or phosphates, and compounds for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal compounds, for example, parabens, chlorobutanol, phenol, ascorbic acid, thimerosal, and the like. In many cases, it will be preferable to include isotonic compounds, for example, sugars, polyalcohols such as manitol, sorbitol, sodium chloride in the composition. Prolonged absorption of the injectable compositions can be brought about by including in the composition a compound, which delays absorption, for example, aluminum monostearate and gelatin.

Sterile injectable solutions can be prepared by incorporating the active compound (e.g., a TDFRP compound and/or additional agent) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding compounds, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating compound such as alginic acid, Primogel, or corn starch; a lubricant such as magnesium stearate or Sterotes; a glidant such as colloidal silicon dioxide; a sweetening compound such as sucrose or saccharin; or a flavoring compound such as peppermint, methyl salicylate, or orange flavoring.

Release agents, wetting agents, coating agents, sweetening, flavoring and perfuming agents, preservatives and antioxidants may also be present in the pharmaceutical compositions. Exemplary coating agents for tablets, capsules, pills and like, include those used for enteric coatings, such as cellulose acetate phthalate, polyvinyl acetate phthalate, hydroxypropyl methylcellulose phthalate, methacrylic acid-methacrylic acid ester copolymers, cellulose acetate trimellitate, carboxymethyl ethyl cellulose, hydroxypropyl methyl cellulose acetate succinate, and the like. Examples of pharmaceutically acceptable antioxidants include: water-soluble antioxidants, such as ascorbic acid, cysteine hydrochloride, sodium bisulfate, sodium metabisulfate sodium sulfite and the like; oil-soluble antioxidants, such as ascorbyl palmitate, butylated hydroxyanisole, butylated hydroxytoluene, lecithin, propyl gallate, alpha-tocopherol, and the like; and metal-chelating agents, such as citric acid, ethylenediamine tetraacetic acid, sorbitol, tartaric acid, phosphoric acid, and the like.

According to some embodiments, the present disclosure is directed to methods and compositions for pulmonary delivery of TDFRP therapeutic compositions comprising penetration enhancers, carrier compounds and/or transfection agents. Methods of pulmonary delivery are disclosed in International Publication WO 99/60166, which is incorporated herein by reference in its entirety.

Briefly, the compounds and methods of the invention employ particles containing TDFRP therapeutics or diagnostics. The particles can be solid or liquid, and can be of respirable size: that is, particles of a size sufficiently small to pass through the mouth and larynx upon inhalation and into the bronchi and alveoli of the lungs. In general, particles ranging from about 5 to 20 microns in size are respirable and are expected to reach the bronchioles (Allen, Secundum Artem, Vol. 6, No. 3, on-line publication updated May 8, 1998, and available at www.paddocklabs.com/secundum/secarndx.html). It is greatly desirable to avoid particles of non-respirable size, as these tend to deposit in the throat and be swallowed, thus reducing the quantity of TDFRP reaching the lung. Liquid pharmaceutical compositions of TDFRPs can be prepared by combining the oligonucleotide with a suitable vehicle, for example sterile pyrogen free water, or saline solution. Other therapeutic compounds may optionally be included.

The present invention also contemplates the use of solid particulate compositions. Such compositions can comprise particles of TDFRPs that are of respirable size. Such particles can be prepared by, for example, grinding dry oligonucleotide by conventional means, for example with a mortar and pestle, and then passing the resulting powder composition through a 400 mesh screen to segregate large particles and agglomerates. A solid particulate composition comprised of a TDFRP can optionally contain a dispersant which serves to facilitate the formation of an aerosol, for example lactose.

In accordance with the methods of the present invention, TDFRP compositions are aerosolized. Aerosolization of liquid particles can be produced by any suitable means, such as with a nebulizer. See, for example, U.S. Pat. No. 4,501,729, incorporated by reference in its entirety herein. Nebulizers are commercially available devices which transform solutions or suspensions into a therapeutic aerosol mist either by means of acceleration of a compressed gas, typically air or oxygen, through a narrow venturi orifice or by means of ultrasonic agitation. Suitable nebulizers include those sold by BLAIREX® under the name PARI LC PLUS, PARI DURA-NEB 2000, PARI-BABY Size, PARI PRONEB Compressor with LC PLUS, PARI WALKHALER Compressor/Nebulizer System, PARI LC PLUS Reusable Nebulizer, and PARI LC Jet+® Nebulizer.

Exemplary formulations for use in nebulizers comprise a TDFRP compound in a liquid, such as sterile, pyrogen free water, or saline solution, wherein the oligonucleotide comprises up to about 40% w/w of the formulation. If desired, further additives such as preservatives (for example, methyl hydroxybenzoate) antioxidants, and flavoring agents can be added to the composition.

TDFRP compounds can also be aerosolized using any solid particulate medicament aerosol generator known in the art. Such aerosol generators produce respirable particles, as described above, and further produce reproducible metered dose per unit volume of aerosol. Suitable solid particulate aerosol generators include insufflators and metered dose inhalers. Metered dose inhalers suitable for used in the art (along with the trade name, manufacturer and indication they are used for) and useful in the present invention include:

### Delivery Device/Trade Name/Manufacturer/Indication

Metered Dose Inhaler (MDI)/Alupent/Boehringer Ingelheim/Beta-adrenergic bronchodilator
Metered Dose Inhaler (MDI)/Atrovent/Boehringer Ingelheim/Anticholinergic bronchodilator
Aerobid/Aerobid-M/Forest/Steriodal Anti-inflammatory
Beclovent/Beconase/Glaxo Wellcome/Steriodal Anti-inflammatory
Flovent/Glaxo Wellcome/Steriodal Anti-inflammatory
Ventolin/Glaxo Wellcome/Beta-adrenergic bronchodilator
Proventil/Key Pharm./Beta-adrenergic bronchodilator
Maxair/3M Pharm./Beta-adrenergic bronchodilator
Azmacort/Rhone-Poulenc Rorer/Steriodal Anti-inflammatory
Tilade/Rhone-Poulenc Rorer/Anti-inflammatory (inhibits release of inflammatory mediators)
Intal/Rhone-Poulenc Rorer/Inhibits mast cell degranulation (Asthma)
Vanceril/Schering/Steriodal Anti-inflammatory
Tornalate/Dura Pharm./Beta-adrenergic bronchodilator

### Solutions for Nebulization

Alupent/Boehringer Ingelheim/Beta-adrenergic bronchodilator
Pulmozyme/Genetech/Recombinant human deoxyribonuclease I
Ventolin/Glaxo Wellcome/Beta-adrenergic bronchodilator
Tornalate/Dura Pharm./Beta-adrenergic bronchodilator
Intal/Rhone-Poulenc Rorer/Inhibits mast cell degranulation (Asthma)

### Capsules (Powder) for Inhalation

Ventolin/Glaxo Wellcome/(Rotocaps for use in Rotohaler device)/Beta-adrenergic bronchodilator

### Powder for Inhalation

Pulmicort/Astra USA/(Turbuhaler device)/Steroidal Anti-inflammatory
   According to some embodiments, liquid or solid aerosols are produced at a rate of from about 10 to 150 liters per minute, from about 30 to 150 liters per minute, or about 60 liters per minute.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared as pharmaceutical compositions in the form of suppositories (e.g., with conventional suppository bases such as cocoa butter and other glycerides) or retention enemas for rectal delivery. The compounds can be prepared for use in conditioning or treatment of ex vivo explants or implants.

Compositions may also be formulated to provide slow or controlled release of the active agent using, by way of example, hydroxypropyl methyl cellulose in varying proportions or other polymer matrices, liposomes and/or microspheres. In addition, the pharmaceutical compositions of the disclosure may contain opacifying agents and may be formulated so that they release the active agent only, or preferentially, in a certain portion of the gastrointestinal tract, optionally, in a delayed manner. Examples of embedding compositions which can be used include polymeric substances and waxes. The active agent can also be in micro-encapsulated form, optionally with one or more of the above-described excipients. According to one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4.522,811, incorporated by reference in its entirety herein.

According to some embodiments, it is advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the disclosure are dictated by and directly dependent on the unique characteristics of the active compound and the particular therapeutic effect to be achieved, and the limitations inherent in the art of compounding such an active compound for the treatment of individuals.

The nucleic acid molecules of the disclosure can be inserted into vectors and used as gene therapy vectors. Gene therapy vectors can be delivered to a subject by, for example, intravenous injection, local administration (see, e.g., U.S. Pat. No. 5,328,470) or by stereotactic injection (see, e.g., Chen, et al., 1994. Proc. Natl. Acad. Sci. USA 91: 3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells that produce the gene delivery system. The pharmaceutical compositions can be included in a container, pack, or dispenser together with instructions for administration.

All publications and patent applications cited in this specification are herein incorporated by reference in their entirety for all purposes as if each individual publication or patent application were specifically and individually indicated to be incorporated by reference for all purposes. The publications discussed herein are provided solely for their disclosure prior to the filing date of the present application. Nothing herein is to be construed as an admission that the inventors described herein are not entitled to antedate such disclosure by virtue of prior disclosure or for any other reason.

### EXAMPLES

### Example 1: Tissue Differentiation Factor Related Polypeptides (TDFRPs) Affected Breast Cancer Stem Cell Self-Renewal In Vitro

### Methods

Cancer stem cells are defined by an inherent capability to undergo self-renewal, to give rise to an aberrantly differentiated progeny, and to seed tumors in vivo (Clevers H. The cancer stem cell: premises, promises and challenges. Nat Med. 2011; 17:313-319.).

To investigate Peptide 123 (SEQ ID NO: 1) and BMP-7 effects on cancer stem cell function, their ability to influence self-renewal in vitro by using the mammosphere assay was examined. The mammosphere assay was developed as a method to propagate mammary epithelial stem cells (MaSC) in vitro by Dontu et al. (Genes Dev 17: 1253-1270 (2003)).

A procedure as described in manufacturer's manual (ProMab, California) was followed to grow cancer stem cells into mammospheres. For mammosphere culture, isolated human breast cancer stem cells were seeded in six well ultralow adherence plates (Corning Inc., Corning, NY, USA) and grown as non-adherent cell suspensions in serum-free stem cell culture medium (ProMab, California). Primary and secondary mammosphere formation was achieved by using weekly trypsinization and dissociation, followed by reseeding in ultralow adherence six well plates.

### Results

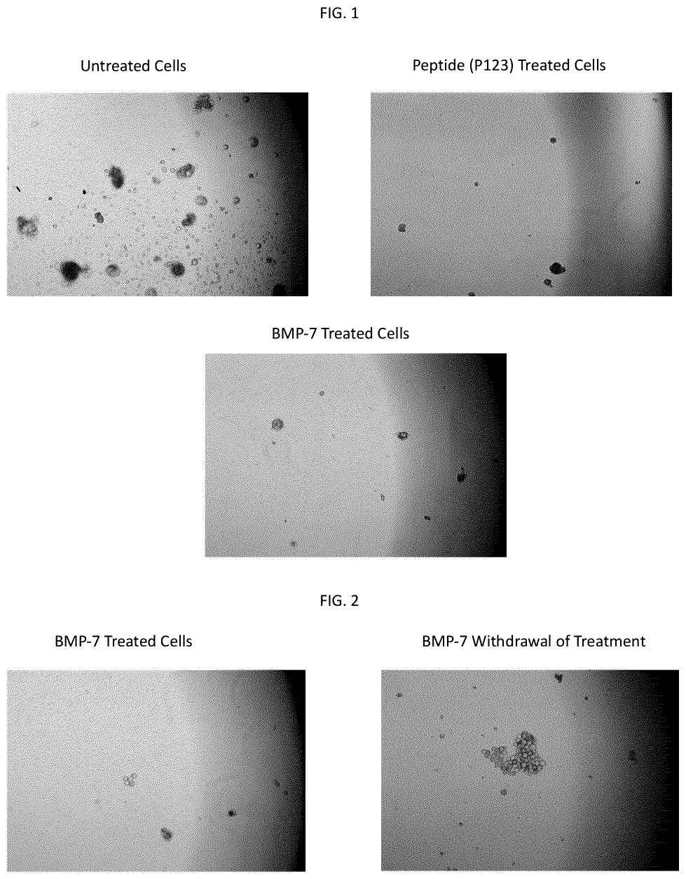

The ability of Peptide 123 and BMP-7 to influence breast cancer stem cell self-renewal in vitro was examined by observing their respective effects on mammosphere formation. As expected, the stem cells when grown in non-adherent cell suspension in serum-free medium formed tumorspheres after 5 to 7 days (FIG. 1). Treatment of these cells with Peptide 123 (300 uM) caused a profound inhibition of tumorsphere formation (FIG. 1). Similarly, BMP-7 (500 ng/ml) treatment also inhibited tumorsphere formation (FIG. 1). After withdrawal of BMP-7 however, these cells regained the ability to form tumorspheres (FIG. 2). Importantly, the tumorsphere formation induced by Peptide 123 is similar to that of BMP-7 (FIG. 1). This suggests that Peptide 123 and BMP-7 both have the ability to inhibit self-renewal and growth of human breast cancer stem cells.

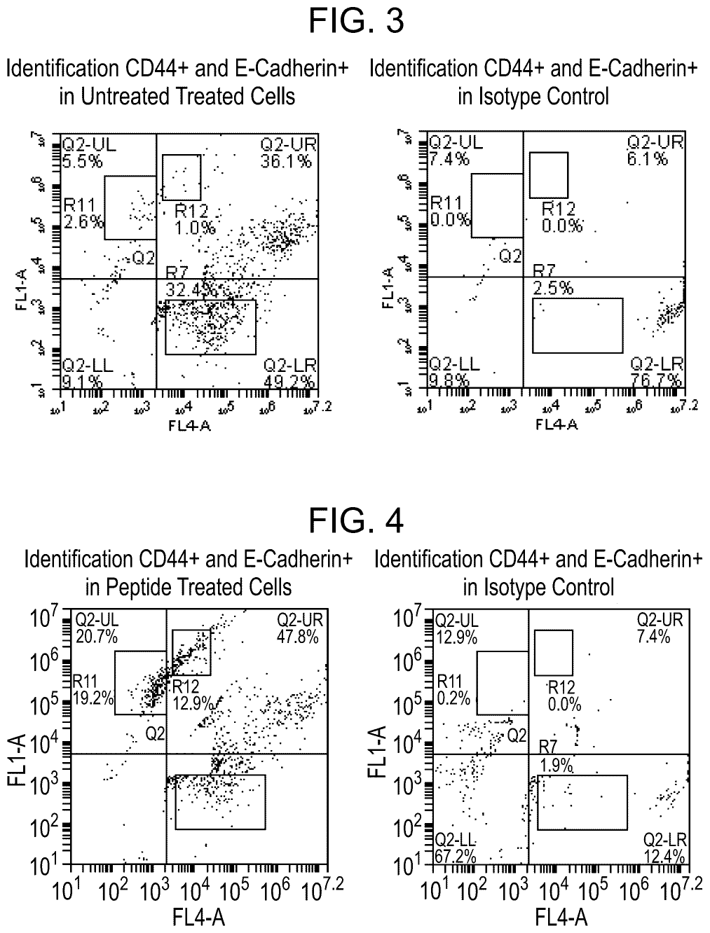

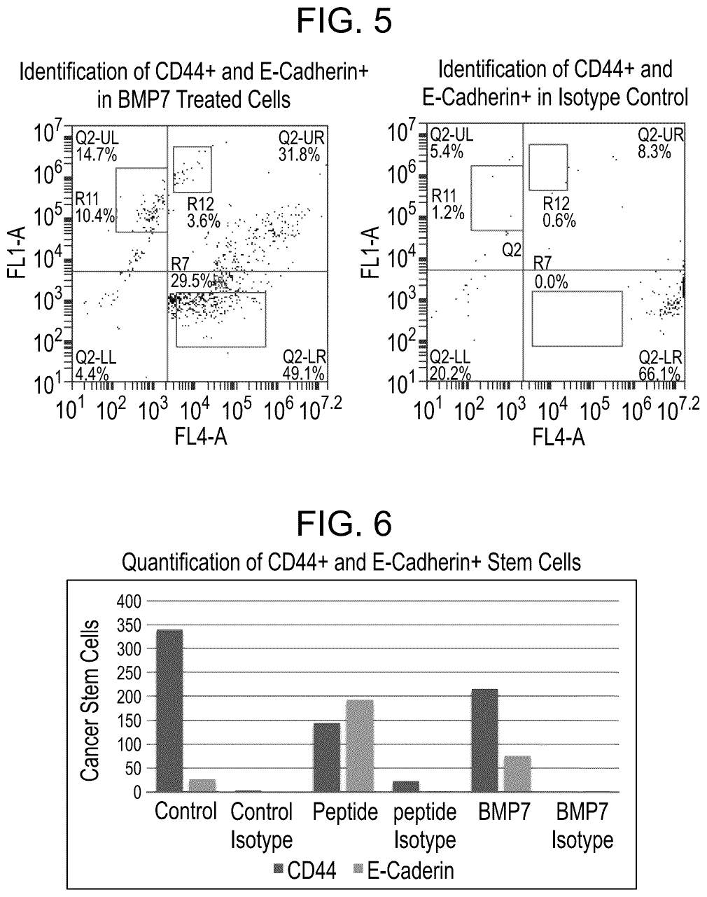

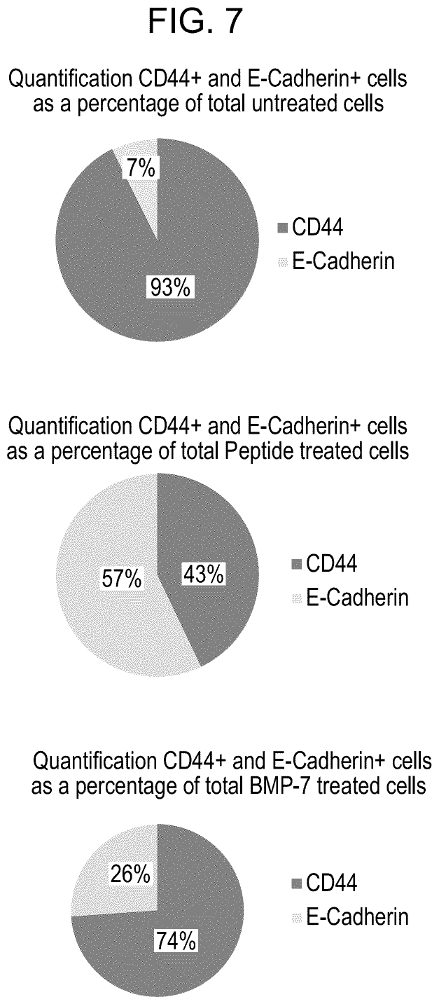

Differentiation of chemo-resistant cancer stem cells, specifically a loss of its stem cell phenotype and self-renewal ability, could be a viable approach to the treatment of tumor recurrence and metastasis (Chirasani S R, Sternjak A, Wend P, et al. Bone morphogenetic protein-7 release from endogenous neural precursor cells suppresses the tumourigenicity of stem-like glioblastoma cells. Brain 2010, 133:1961-1972). Although this approach may or may not directly kill the cancer cells, it could make CSCs more responsive to conventional chemotherapies. Therefore, Peptide 123 and BMP-7 were examined to determine if they affected differentiation of human breast cancer stem cells. Sensitive flow cytometry method (Gupta V et al., Methods Mol Biol. 2011, 716: 179-191) to quantify CD44+, indicating stem cell phenotype, and E-cadherin+, indicating epithelial phenotype cells, after treatment with or without P123 (SEQ ID NO: 1) and BMP-7. Human breast cancer stem cells were grown in non-adherent cell suspension in serum-free medium for 8 Days. FACS analysis showed that a majority of cells (93%) were CD44+ cells, with a minority (7%) being MCF-7 cells from which the cancer stem cells were isolated (FIG. 3). Cells treated with P123 (300 uM) or BMP-7 (314-430 ng/ml), showed a marked decline in CD44+ cells (FIGS. 4 and 5). Importantly, the percent loss of CD44+ cells after treatment with the P123, 50%, was greater than that after treatment with BMP-7, 19% (FIGS. 4 and 5). Furthermore, a gain in E-cadherin+ cells was observed, which was proportional to the loss of CD44+ cells (FIGS. 4-6). Similarly, the percent gain in E-cadherin+ cells after treatment with P123, 50%, was greater than that after treatment with BMP-7, 19%, (FIG. 7). These results suggested that Peptide 123 and BMP-7 had the ability to induce a loss of stem cell phenotype of human breast cancer stem cells and may reverse EMT by promoting epithelial differentiation of cancer stem cells.

The initial results demonstrate that Peptide 123, a peptide agonist of BMP signaling, and BMP-7, had the ability to inhibit self-renewal and growth of human breast cancer stem cells. In addition, these compounds had the potential to reverse EMT by inducing a loss of stem cell phenotype and promoting epithelial differentiation.

These findings extend those from studies that Peptide 123 and BMP, are capable of inhibiting bulk tumor cell growth from prostate cancer by activating SMAD 1/5/8 signaling and controlling its cell cycle pathway (Bosukonda, A et al., BIOLOGY OF REPRODUCTION 81: 372. (2009) Bone Morphogenetic Protein-6 (BMP-6) Efficacy and Its Mechanism of Action to Suppress Human Prostate Cancer Cell Growth.). Together, these studies demonstrate that Peptide 123, a peptide analogue based on BMP signaling, shows therapeutic potential for suppressing bulk tumor cells and CSCs. In conclusion, Peptide 123 appears to be a promising therapeutic in a new class of drugs that can eliminate the primary tumor and prevent reoccurrence.

### REFERENCES

1. Muhammad Al-Hajj and Michael F Clarke. Self-renewal and solid tumor stem cells Oncogene (2004) 23, 7274-7282.
2. Mani, S. A., Guo, W., Liao, M. J., Eaton, E. N., Ayyanan, A., Zhou, A. Y., Brooks, M., Reinhard, F., Zhang, C. C., Shipitsin, M., et al., 2008. The epithelial-mesenchymal transition generates cells with properties of stem cells. Cell. 2008; 133:704-715.
3. Hollier B. G., Evans K., Mani S. A. The epithelial-to-mesenchymal transition and cancer stem cells: A coalition against cancer therapies. J. Mammary. Gland. Biol. Neoplasia. 2009;14:29-43
4. Santisteban M., Reiman J. M., Asiedu M. K., Behrens M. D., Nassar A., Kalli K. R., Haluska P., Ingle J. N., Hartmann L. C., Manjili M. H., Radisky D. C., Ferrone S., Knutson K. L. Immune-induced epithelial to mesenchymal transition in vivo generates breast cancer stem cells. Cancer Res. 2009, 69:2887-2895.
5. Hugo H., Ackland M. L., Blick T., Lawrence M. G., Clements J. A., Williams E. D., Thompson E. W. Epithelial-mesenchymal and mesenchymal-epithelial transitions in carcinoma progression. J. Cell Physiol. 2007;213:374-383
6. Wang Z., Li Y., Ahmad A., Azmi A. S., Kong D., Banerjee S., Sarkar F. H. Targeting miRNAs involved in cancer stem cell and EMT regulation: An emerging concept in overcoming drug resistance. Drug Resist. Updat. 2010;13:109-118
7. Singh A., Settleman J. EMT, cancer stem cells and drug resistance: An emerging axis of evil in the war on cancer. Oncogene. 2010;29:4741-4751
8. Deonarain et al., MAbs. 2009 January-February; 1(1): 12-25
9. Gupta, P. B., Chaffer, C. L., Weinberg, R. A., 2009. Cancer stem cells: mirage or reality? Nat. Med. 15, 1010-1012.
10. Bosukonda, A et al., BIOLOGY OF REPRODUCTION 81: 372. (2009) Bone Morphogenetic Protein-6 (BMP-6) Efficacy and Its Mechanism of Action to Suppress Human Prostate Cancer Cell Growth.
11. Clevers H. The cancer stem cell: premises, promises and challenges. Nat Med. 2011; 17:313-319.
12. Chirasani S R, Sternjak A, Wend P, et al. Bone morphogenetic protein-7 release from endogenous neural precursor cells suppresses the tumourigenicity of stem-like glioblastoma cells. Brain 2010, 133:1961-1972.
13. Gupta V et al., Methods Mol Biol. 2011, 716: 179-191.

1. A method of treating a subject having cancer, the method comprising administering one or more tissue differentiation factor related polypeptides (TDFRPs) and one or more antineoplastic modalities to the subject, thereby rendering a cancer cell responsive to the one or more antineoplastic modalities, and thereby treating the cancer in the subject.

2. The method of claim 1, wherein the subject is administered the one or more antineoplastic modalities before, after, before and after, or concurrently with the TDFRP.

3. (canceled)

4. (canceled)

5. The method of claim 1, wherein the one or more TDFRPs comprises an amino acid sequence at least 90%, 95%, 96%, 97%, 98% or 99% identical to SEQ ID NO: 1 or 3, wherein the one or more TDFRPs comprises a crosslink, or the one or more TDFRPs do not comprise a crosslink.

6. (canceled)

7. The method of claim 1, wherein the cancer cell expresses bone morphogenetic protein (BMP) receptors.

8. The method of claim 1, wherein the cancer is selected from the group consisting of: breast cancer, prostate cancer, renal cell carcinoma, bone metastasis, lung cancer or metastasis, osteosarcoma, multiple myeloma, astrocytoma, pilocytic astrocytoma, dysembryoplastic neuroepithelial tumor, oligodendrogliomas, ependymoma, glioblastoma multiforme, mixed gliomas, oligoastrocytomas, medulloblastoma, retinoblastoma, neuroblastoma, germinoma, teratoma, gangliogliomas, gangliocytoma, central gangliocytoma, primitive neuroectodermal tumors (PNET, e.g. medulloblastoma, medulloepithelioma, neuroblastoma, retinoblastoma, ependymoblastoma), tumors of the pineal parenchyma (e.g. pineocytoma, pineoblastoma), ependymal cell tumors, choroid plexus tumors, neuroepithelial tumors of uncertain origin (e.g. gliomatosis cerebri, astroblastoma), esophageal cancer, colorectal cancer, CNS, ovarian, melanoma pancreatic cancer, squamous cell carcinoma, hematologic cancer (e.g., leukemia, lymphoma, and multiple myeloma), colon cancer, rectum cancer, stomach cancer, kidney cancer, mesothelioma, bladder cancer, skin cancer, and a combination thereof.

9. (canceled)

10. The method of claim 1, wherein the cancer cell is resistant to the one or more antineoplastic modalities.

11. (canceled)

12. The method of claim 1, wherein the one or more antineoplastic modalities are one or more chemotherapeutic agents, surgery, radiation, immunotherapy, hormone therapy, stem cell transplant, small-molecules, antibodies, chimeric antigen receptor T cells (CAR-T cells), cancer vaccines, or a combination thereof.

13. A method of producing mature dendritic cells, the method comprising:
a) providing monocytes from a subject having cancer;
b) contacting the monocytes with one or more tissue differentiation factor related polypeptides (TDFRPs), thereby producing immature dendritic cells;
c) contacting the immature dendritic cells with one or more TDFRPs, thereby producing mature dendritic cells; and
d) isolating mature dendritic cells.

14. The method of claim 13, wherein
step b) further comprises contacting the monocytes with interleukin 4 (IL-4) and Granulocyte-macrophage colony-stimulating factor (GM-CSF); and/or
step c) further comprises contacting the immature dendritic cells with lipopolysaccharide; and/or
step b) comprises culturing the monocytes with one or more TDFRPs for about 3 to about 5 days; and/or
step c) comprises culturing the immature dendritic cells with one or more TDFRPs for about 24 hours to about 48 hours.

15.-23. (canceled)

24. An isolated population of mature dendritic cells (DCs) produced by the method of claim 13.

25. An isolated population of mature dendritic cells (DCs), wherein the mature dendritic cells are differentiated from monocytes from a subject having cancer, and wherein the monocytes are contacted with one or more tissue differentiation factor related polypeptides (TDFRPs), thereby producing mature dendritic cells.

26. The isolated population of mature dendritic cells of claim 25, wherein the one or more TDFRPs comprises an amino acid sequence at least 90%, 95%, 96%, 97%, 98% or 99% identical to SEQ ID NO: 1 or 3, wherein the wherein the one or more TDFRPs comprises a crosslink, or the one or more TDFRPs do not comprise a crosslink.

27. (canceled)

28. The isolated population of mature dendritic cells of claim 25, wherein the monocytes are first contacted with interleukin 4 (IL-4) and Granulocyte-macrophage colony-stimulating factor (GM-CSF) and the one or more TDFRPs, thereby producing immature dendritic cells; and wherein the immature dendritic cells are contacted with lipopolysaccharide.

29.-31. (canceled)

32. The isolated population of mature dendritic cells of claim 25, wherein the cancer comprises cancer cells expressing bone morphogenetic protein (BMP) receptors.

33. The isolated population of mature dendritic cells of claim 25, wherein the cancer is selected from the group consisting of: breast cancer, prostate cancer, renal cell carcinoma, bone metastasis, lung cancer or metastasis, osteosarcoma, multiple myeloma, astrocytoma, pilocytic astrocytoma, dysembryoplastic neuroepithelial tumor, oligodendrogliomas, ependymoma, glioblastoma multiforme, mixed gliomas, oligoastrocytomas, medulloblastoma, retinoblastoma, neuroblastoma, germinoma, teratoma, gangliogliomas, gangliocytoma, central gangliocytoma, primitive neuroectodermal tumors (PNET, e.g. medulloblastoma, medulloepithelioma, neuroblastoma, retinoblastoma, ependymoblastoma), tumors of the pineal parenchyma (e.g. pineocytoma, pineoblastoma), ependymal cell tumors, choroid plexus tumors, neuroepithelial tumors of uncertain origin (e.g. gliomatosis cerebri, astroblastoma), esophageal cancer, colorectal cancer, CNS, ovarian, melanoma pancreatic cancer, squamous cell carcinoma, hematologic cancer (e.g., leukemia, lymphoma, and multiple myeloma), colon cancer, rectum cancer, stomach cancer, kidney cancer, mesothelioma, bladder cancer, skin cancer, and a combination thereof.

34. (canceled)

35. A method of treating a subject having cancer, comprising administering to the subject an effective amount of the isolated population of mature dendritic cells of claim 25.

36.-37. (canceled)

38. The method of claim 36, wherein the one or more TDFRPs comprises an amino acid sequence at least 90%, 95%, 96%, 97%, 98% or 99% identical to SEQ ID NO: 1 or 3, wherein the wherein the one or more TDFRPs comprises a crosslink, or the one or more TDFRPs do not comprise a crosslink.

39. (canceled)

40. The method of claim 35, wherein the mature dendritic cells are administered to the same subject from which the monocytes were obtained or the mature dendritic cells are administered to a different subject than the subject from which the monocytes were obtained

41. (canceled)

42. The method of claim 35, wherein the cancer is selected from the group consisting of: breast cancer, prostate cancer, renal cell carcinoma, bone metastasis, lung cancer or metastasis, osteosarcoma, multiple myeloma, astrocytoma, pilocytic astrocytoma, dysembryoplastic neuroepithelial tumor, oligodendrogliomas, ependymoma, glioblastoma multiforme, mixed gliomas, oligoastrocytomas, medulloblastoma, retinoblastoma, neuroblastoma, germinoma, teratoma, gangliogliomas, gangliocytoma, central gangliocytoma, primitive neuroectodermal tumors (PNET, e.g. medulloblastoma, medulloepithelioma, neuroblastoma, retinoblastoma, ependymoblastoma), tumors of the pineal parenchyma (e.g. pineocytoma, pineoblastoma), ependymal cell tumors, choroid plexus tumors, neuroepithelial tumors of uncertain origin (e.g. gliomatosis cerebri, astroblastoma), esophageal cancer, colorectal cancer, CNS, ovarian, melanoma pancreatic cancer, squamous cell carcinoma, hematologic cancer (e.g., leukemia, lymphoma, and multiple myeloma), colon cancer, rectum cancer, stomach cancer, kidney cancer, mesothelioma, bladder cancer, skin cancer, and a combination thereof.

43. An in vivo method of increasing T-cell recognition of tumor cell antigens on tumor cells in a subject having cancer, comprising administering to the subject an effective amount of the isolated population of mature dendritic cells of claim 24, wherein TGF-beta secretion from the tumor cells is decreased compared to a control tumor cell.
